(19) United States
(12) Patent Application Publication
(10) Pub. No.: US 2023/0125012 A1
(43) Pub. Date: Apr. 20, 2023

(54) SYSTEMS AND METHODS FOR INTRODUCING A STENT-GRAFT THROUGH A BLOOD VESSEL LOCATED ABOVE A DIAPHRAGM

(71) Applicant: MAJOR MEDICAL DEVICES, INC., New York, NY (US)

(72) Inventors: Andrew KERR, New York, NY (US); David C. MAJERCAK, Bloomington, IN (US)

(21) Appl. No.: 17/801,084

(22) PCT Filed: Feb. 19, 2021
(86) PCT No.: PCT/US2021/018777

§ 371 Date: Aug. 19, 2022

Related U.S. Application Data
(60) Provisional application No. 62/979,008, filed on Feb. 20, 2020.
(60) Provisional application No. 63/067,678, filed on Aug. 19, 2020.

(51) Int. Cl. A61F 2/07; A61F 2/89; A61F 2/954; A61F 2/95

(52) U.S. Cl. CPC A61F 2/07; A61F 2/89; A61F 2/954; A61F 2/9522

(57) ABSTRACT

Systems and methods for repairing aneurysms (e.g., abdominal aneurysm) are provided. The systems and methods provide stent-graft systems having a first stent and a main graft body wherein the main graft body is configured to be inserted through a blood vessel located above a diaphragm of the patient into a target blood vessel. In some instances, the first stent and the main graft body can be in a substantially end-to-end configuration. In some instances, the stent-graft systems and methods are configured for use in a single arterial puncture or incision in, for example, a blood vessel with a diameter less than or equal to the diameter of a femoral artery from the same patient or subject.

All references cited herein, including but not limited to patents and patent applications, are incorporated by reference in their entirety.

### BACKGROUND

An aneurysm is an abnormal enlargement or bulge in a blood vessel. Aortic aneurysms can cause embolization into branch vessels, aortic thrombosis, and aortic rupture. Damaged blood vessels can be treated or repaired by surgery or by endovascular graft placement.

The aorta is the largest artery in the body originating from the left ventricle of the heart and extending into the abdomen. It bifurcates into the two iliac arteries. Arterial aneurysms (AA) occur in every part of the aorta and its branches. Sabiston Textbook of Townsend, et al., Surgery: The Biological Basis of Modern Surgical Practice 20th Edition, pp.1722-1753 (2017).

AAs are typically repaired with open surgery or endovascular aneurysm repair (EVAR). EVAR is touted as a minimally invasive procedure that utilizes stent-graft devices to repair an AA. A stent-graft device is a combination device that includes a stent portion and a graft portion connected to each other such that they can be deployed together to repair damage to a blood vessel.

A stent is typically an expandable metal lattice device inserted into a blood vessel and expanded to open a constricted, damaged or occluded blood vessel. In addition to opening the blood vessel, a stent can provide a rigid structural support to prevent the blood vessel from re-closing. Stents are often used together with balloon angioplasty.

A prosthetic graft is a medical device that can be used to replace or repair a diseased blood vessel. The graft can be made of a synthetic material (e.g., ePTFE, polyester) that can be expanded to approximate the diameter of the blood vessel in need of repair. The graft material provides a blood-tight seal such that it can support normal blood flow without leakage.

A stent-graft device can provide a combination of substantially blood-tight seal from the graft with the support structure of a stent to prevent the stent-graft device from dislodgment under the pressure of normal blood flow. The combination of the stent with the prosthetic graft can be used to hydraulically isolate an aneurysm when positioned across the neck of the aneurysm. However, connecting and deploying components of stent-graft devices has proved to be challenging because the delivery system profile (i.e., outer diameter of the delivery sheath) of a stent-graft device can be significant, making the device more difficult to insert, to navigate the target anatomy, and to deploy. In order to address these issues, several low-profile stent-graft devices have been developed by an inventor of the present application including, for example, as described in U.S. Pat. Nos. 10,105,209; 9,050,182; 8,257,423; 7,105,017; 7,175,651; 6,981,982; 6,015,422; 6,102,918; and 6,168,620.

AAs are often asymptomatic and frequently occur in people over the age of 65 years. The mortality rate of AAs, if untreated, is high and as such, early detection and repair is important. EVAR is preferred to open surgery as a less invasive alternative for those aortic aneurysm patients who are appropriate candidates. Although safer, faster, and less invasive than open surgical repair, EVAR can still be difficult, lengthy, and produce post procedural morbidity.

The EVAR procedure for infrarenal aortic aneurysm typically requires one puncture or incision in each of the femoral arteries followed by maneuvering the constrained stent-graft device in an upward, cephalad direction (femoral approach) into position in the infrarenal aorta over a guidewire constrained in an introducer sheath. A portion of the stent-graft device is deployed in the infrarenal aorta. Next, the stent-graft device is positioned and deployed in one iliac artery. Another stent-graft device is then positioned and deployed in the other iliac artery second via the puncture or incision in the other femoral artery. Such procedures rely on inserting and deploying stent-graft devices from below the diaphragm of a subject or patient.

As summarized, the standard EVAR procedure requires bilateral femoral punctures (i.e., one puncture or incision in each femoral artery) for arterial access, in addition to making fine positioning movements for the stent-graft device from a femoral approach orientation (from below the diaphragm). While superior to open surgery, EVAR, as described above, requires patient recovery from the bilateral femoral punctures used for access. Patients often remain at bedrest for several days, require pain medication, and thereby incur additional costs (e.g., hospital stay, medication, loss of time at work).

The delivery system profile of currently available stent-graft devices restricts the blood vessels that can be a target for introduction of these devices. Such devices have a wide diameter due, in part, to how the components of the stent-graft device are arranged in catheter based systems used for their delivery and/or connected to each other.

What is needed are improved low-profile stent-graft systems and methods of repairing aneurysms that are lower in profile and, as such, easier to deploy in lower diameter blood vessels and with less post procedure morbidity and patient discomfort than currently available stent-graft devices.

### SUMMARY

Stent-graft systems and methods described herein provide, in some aspects, low profile stent-graft devices configured to be introduced/inserted in a blood vessel located above the diaphragm of the patient and deployed in a “top down” approach. Exemplary systems and methods for adjusting the placement of stent-graft devices in a target blood vessel (e.g., infrarenal aorta, juxtarenal aorta, pararenal aorta, thoracic aorta, or suprarenal aorta) after an initial deployment are also provided. Further aspects describe a centering device for use in centering the deployed location of stent-graft devices in a target blood vessel.

One aspect described herein is directed to a first stent-graft system for repair of an aneurysm in a target blood vessel having a first stent, and a main graft body wherein the stent-graft system can be configured to be inserted through a single arterial puncture or incision in an insertion site blood vessel located above a diaphragm of a patient.

Another aspect provides a first method of repairing an abdominal aortic aneurysm in a patient, by puncturing an insertion site blood vessel located above a diaphragm of the patient and creating a passage in the insertion site blood vessel. The stent-graft system can be inserted in the passage of the insertion site blood vessel. The stent-graft system can comprise a main graft body that bifurcates into a first limb gate and a second limb gate. The main graft body of the stent graft system can be positioned and deployed, for example, in a target blood vessel of the patient.

Further aspects provide a second stent-graft system (e.g., an endograft deployment system) for deploying an endograft in a target blood vessel of a patient or subject. In some instances of the second stent-graft system, the endograft can be deployed from below the diaphragm of a patient or subject. The endograft deployment system can have an outer tube comprising a central inner member comprising an endograft and a carrier tube comprising a tether wire. The tether wire can have a caudad end and a more cephalad portion (i.e., end closest to the head or top of the body). The endograft deployment system can have a top stent surrounding the central inner member, wherein the top stent comprises a plurality of hooks having a plurality of receptacles and a plurality of sutures.

In some instances of the second stent-graft system, a first end of at least a first suture can be disposed through one of the plurality of receptacles for retaining the top stent in a constrained configuration. A second end of the first suture can be affixed to the more cephalad portion of the tether wire. Movement of the tether wire can control removal of the plurality of sutures from the plurality of receptacles, release the top stent from a constrained to an unconstrained configuration, and remove the plurality of sutures from the blood vessel of the patient or subject.

One aspect described herein provides a second method of deploying a stent-graft in a patient or subject, by puncturing in a first femoral artery and a second femoral artery and creating a passage in each of the femoral arteries and inserting a stent-graft in a passage of either the first femoral artery or the second femoral artery with a stent-graft deployment system comprising an outer tube comprising a central inner member comprising an endograft and a carrier tube comprising a tether wire. In some instances of the second method, the endograft can be deployed from above the diaphragm or below the diaphragm of a patient or subject.

In some instances of the second method, the tether wire has a caudad end and a more cephalad portion. A top stent can surround the central inner member, wherein the top stent comprises a plurality of hooks having a plurality of receptacles. Some instances of the second method can further include a plurality of sutures, wherein a first end of at least a first suture is disposed through one of the plurality of receptacles for retaining the top stent in a constrained configuration, and a second end of the first suture is affixed to the more cephalad portion of the tether wire.

The tether wire can be moved to remove the plurality of sutures from the plurality of receptacles, release the top stent from a constrained to an unconstrained configuration, and remove the plurality of sutures from a blood vessel of the patient or subject. In some instances, the tether wire is removed, and the sutures can be left in the patient.

Aspects described herein provide a third stent-graft system for repair of an aneurysm in a target blood vessel comprising a main graft body comprising a sealing stent at least partially disposed in the main graft body, wherein the stent-graft system can be configured to be inserted through a single arterial puncture or incision in an insertion site blood vessel located above a diaphragm of a patient. The main-graft body is configured to be inserted through a single arterial puncture or incision in an insertion blood vessel located above a diaphragm of a patient.

Further aspects described herein provide a third method of positioning a main graft body of a stent-graft system in a target blood vessel (e.g., infrarenal aorta, juxtarenal aorta, pararenal aorta, thoracic aorta, or suprarenal aorta) of a subject by advancing a main graft body delivery system to a target location in the target blood vessel. The main graft body delivery system can include the main body graft and a centering device. The main graft body can be positioned in the target blood vessel in a first position at the target location and it can be determined if the first position of the main graft body is centered in the target blood vessel at the target location. In some instances, the third method can be directed to inserting the main graft body through a single arterial puncture or incision in an insertion blood vessel located above a diaphragm of a patient.

The centering device can be deployed in the target blood vessel in a centered position if the first position of the main graft body is not centered in the target blood vessel at the target location. The main graft body can be repositioned in the target blood vessel in a second position determined from the centered position if the first position of the main graft body is not centered in the target blood vessel at the target location.

Aspects described herein provide a fourth stent-graft system for repair of an aneurysm in a target blood vessel, comprising a top stent having a plurality of positioning receptacles and a main graft body wherein the top stent and the main graft body are in a substantially end-to-end configuration. The top stent and the main graft body can be disposed around an inner member. The stent-graft system includes a snare tube comprising a snare loop. A first end of the snare loop can be disposed in the snare tube. A second end of the snare loop can be disposed from the snare tube, through the positioning receptacles, around the inner member, and into the snare tube. The snare tube can be parallel to the inner member and a first end of the snare loop can be adjacent to the second end of snare loop. The stent-graft system can be configured to be inserted through a single arterial puncture or incision in an insertion site blood vessel located above a diaphragm of the patient.

Further aspects provide a fifth stent-graft system for repair of an aneurysm in a target blood vessel, comprising a top stent having a plurality of positioning receptacles and a main graft body wherein the top stent and the main graft body are in a substantially end-to-end configuration and wherein the top stent and the main graft body are disposed around an inner member. The stent-graft system can include a snare loop disposed through the positioning receptacles with a degree of rotation around the top stent of greater than 360 degrees. A first end of the snare loop can be disposed substantially symmetrically with respect to the inner member. The stent-graft system can be configured to be inserted through a single arterial puncture or incision in an insertion site blood vessel located above a diaphragm of the patient. In another aspect, the main-graft body is configured to be inserted through a single arterial puncture or incision in an insertion blood vessel located above a diaphragm of a patient.

### BRIEF DESCRIPTION OF DRAWINGS

FIG. 1 is an exemplary drawing of the descending thoracic aorta, suprarenal abdominal aorta, renal arteries, infrarenal aorta, and the ipsilateral and contralateral iliac arteries;

FIG. 2A shows a front view and FIG. 2B shows a flattened side view of an exemplary stent-graft device in accordance with aspects described herein;

FIG. 3A shows an exemplary top stent portion of an exemplary stent-graft device, including connectors and receptacles;

FIG. 3B shows a close up of exemplary connector and receptacle portions connecting a top stent to a connecting ring in an exemplary stent-graft device;

FIGS. 3C and 3D show alternative configurations for the exemplary connectors and receptacles;

FIG. 4 shows an alternative configuration of the arms of an exemplary connecting ring;

FIG. 5 shows an exemplary configuration of an iliac leg component;

FIGS. 6A-6B show femoral positioning and deployment of a stent-graft device in a typical EVAR procedure;

FIGS. 7A-7D shows an exemplary deployment of a stent-graft device in accordance with aspects described herein wherein said device is inserted/introduced from an access point above the diaphragm (e.g., the axillary or brachial artery);

FIG. 8A shows an exemplary fine positioning of a main body of a stent-graft device in accordance with aspects described herein and FIG. 8B shows a close up view of the example of FIG. 8A;

FIG. 9 shows an alternative stent-graft deployment system that ensures removal of sutures used to guide the introduction and positioning of an exemplary stent-graft;

FIG. 10 shows a top-down view of an optional shelf for use with the alternative stent-graft deployment system shown in FIG. 9;

FIG. 11 shows an exemplary stent-graft device having a snare loop in a snare tube disposed asymmetrically around an inner member for adjusting the axial position of the stent-graft system in a blood vessel;

FIG. 12A shows the exemplary stent-graft device of FIG. 11 with a revealed optional centering device;

FIG. 12B illustrates an alternate embodiment of the device of FIG. 12A;

FIG. 13 shows an exemplary stent-graft device with a snare loop disposed through eyelets on a top stent with a degree of rotation of 540 degrees and snare loop ends disposed symmetrically around an inner member;

FIG. 14 shows cross sectional views of the exemplary devices of FIGS. 11 and 12A; and

FIG. 15 shows cross sectional views of the exemplary device of FIG. 13.

### DETAILED DESCRIPTION

Stent-graft systems and methods are provided herein to improve the introduction, positioning, and deployment of stent-graft devices for repair of aneurysms. It is understood that stent-graft devices in accordance with aspects described herein can be used to repair aneurysms in any suitable blood vessel where access to the blood vessel is through arteries located below the diaphragm.

Contemporary stent-graft systems have an outer diameter of 14-24F (French) and are used in EVAR procedures via insertion of the stent-graft system from below the diaphragm. In current, typical EVAR procedures, two punctures or incisions are made - one in each femoral artery. Cannulation of each puncture is followed by positioning and deployment of a guidewire through each puncture, requiring additional time and increasing the potential risk of variation and error. In addition, closure of said punctures in both femoral arteries can be painful, increasing morbidity issues related to the EVAR procedure and thereby protracting a patient’s recovery.

Thus, in accordance with aspects described herein, stent-graft devices (or components thereof) are configured to be inserted through a single puncture or incision above the diaphragm of a patient or subject (e.g., in a smaller caliber artery). As described herein, these exemplary stent-graft devices can have a smaller diameter (e.g., ranging between about 6 to about 13 French or about 13 to about 22 French outer diameter profile) and be positioned in, for example, the infrarenal aorta in a “top down” orientation for easier and faster positioning and deployment in the infrarenal aorta and the left and right iliac artery. The term “top down” refers to introducing or inserting a stent-graft device in an insertion blood vessel located above a diaphragm of a patient as described herein.

The “top down” approach is expected to substantively reduce the access time and challenges posed by the typical bilateral femoral artery approach since the bilateral approach requires access and instrumentation of devices in both legs of the patient and arteries in the legs of patient’s suffering from abdominal aneurysms (for example) are often highly angulated and diseased making the set up of an EVAR procedure significantly more complex for interventionalists. In this aspect, the procedure is faster, less prone to error, and results in patient recovery from a puncture or incision in an artery located above the diaphragm of the patient. The access location (above the diaphragm) traverses many of the anatomical challenges of the typical EVAR procedure. Aspects described herein provide less tortuosity and lower risk of disease associated with the cannulation and placement of the necessary access devices/instruments that enable the EVAR procedure. Using the aspects described herein, the patient will also have less discomfort and a faster recovery, since “top down” access can be achieved using a single puncture (above the diaphragm access) technique contemplated herein.

One aspect described herein provides a first stent-graft system for repair of an aneurysm in a target blood vessel of a patient comprising a first stent, and a main graft body. In some instances of the first stent-graft system, the stent-graft system can be configured to be inserted through a single arterial puncture or incision in an insertion site blood vessel located above the diaphragm of a subject or patient. In one aspect, the insertion site blood vessel has a diameter less than or equal to the diameter of a femoral artery of the patient.

The term “insertion site blood vessel” refers to a blood vessel into which a stent-graft device is initially inserted during a procedure for repair of a blood vessel (e.g., EVAR procedure).

The term “target blood vessel” refers to a blood vessel that is the site of repair for a disease or conditions (e.g., aortic aneurysm). The target blood vessel can include, but is not limited to, the infrarenal aorta, the juxtarenal aorta, the pararenal aorta, the thoracic aorta, or the suprarenal aorta.

The term “stent” refers to a mesh or lattice tube structure made of, for example, super-elastic nitinol thin wire or laser cut from tubing (or any other suitable material, for example, titanium, or chromium cobalt alloy), that can be inserted into a blood vessel in a constrained state and deployed in an unconstrained state.

The term “constrained” refers to a configuration of the stent-graft system, or components of the stent-graft system, such that its diameter is at a minimum or smaller amount compared to a fully expanded or “unconstrained” configuration of the stent-graft system, or components thereof. The constrained configuration of the stent-graft system can be at a small enough diameter to be introduced into an insertion site blood vessel located above the diaphragm and/or having a diameter less than or equal to the diameter of a femoral artery of the patient.

In some embodiments of the first stent-graft system, the target blood vessel is selected from the group consisting of an infrarenal aorta, a juxtarenal aorta, a pararenal aorta, a thoracic aorta, or a suprarenal aorta.

In some embodiments of the first stent-graft system, the first stent and the main graft body are in a substantially end-to-end configuration. In some embodiments of the first stent-graft system, the insertion site blood vessel is located above a diaphragm of the patient. In another embodiment of the first stent-graft device, the diameter of the stent-graft system in a constrained configuration can be from about 13 to about 22 French. In another embodiment, the diameter of the stent-graft system in a constrained configuration can be from about 6 to about 13 French.

In a further embodiment of the first stent-graft device, the main graft body comprises a densified material. In some instances, the first stent is encapsulated within a densified material (e.g., at least one layer or two layers of polytetrafluoroethylene or ePTFE). In some instances, the ePTFE is substantially free of pores.

The term “densified material” refers to a material (e.g., ePTFE) that has been modified to increase its density in comparison to the same material that has not been modified. For example, a densified material can have an increased density of at least 5, 10, 15, 20, 25, 30, 35, 40, 45, 50, 55, 60, 65, 70, 75, 80, 85, 90, or 95 percent. As an example, ePTFE may be densified by the application of mechanical, compressive force.

A densified material has same or greater tensile strength than a non-densified material and can be thinner than a material that has not been densified. As described herein, a densified material can be used to make graft material than can be compressed to a diameter suitable for insertion into an insertion site blood vessel location above a diaphragm of a patient or subject or in a blood vessel with a diameter less than or equal to the diameter of a femoral artery from the same patient or subject.

In some instances, the insertion site blood vessel (i.e., blood vessel located above a diaphragm of the patient, small blood vessel) is selected from the group consisting of the brachial, radial, ulnar, axillary, and carotid arteries. In some instances, the insertion site blood vessel is an axillary artery. In one aspect, the diameter of the insertion site blood vessel or small blood vessel is 2 to 8 mm. In another example, the insertion site blood vessel is the subclavian artery.

In an alternative embodiment, a guidewire can be introduced into an insertion site blood vessel located above a diaphragm of a subject or patient into a target blood vessel such as the infrarenal aorta. Catheters and/or wires can be inserted into branch vessels (e.g., superior mesenteric artery, celiac artery, renal arteries, and inferior mesenteric artery), and a fenestrated graft can be advanced into a target blood vessel such as the aorta over the catheters and/or wires with each wire coming out of a fenestration and going into one of the branches.

Alternatively, a fenestrated graft can be introduced into an insertion site blood vessel located above a diaphragm of a subject or patient into a target blood vessel such as the infrarenal aorta with no wires going through the fenestrations of the fenestrated graft. The branch vessels can optionally be catheterized.

Another alternative, wires will protrude from each fenestration inside the sheath when the device is loaded, but the branch vessels may not be catheterized until after the main body device is deployed

Some embodiments of the first stent-graft system include a connecting ring comprising a plurality of connectors adapted to receive the plurality of receptacles. The connecting ring can be disposed in the main graft body. The plurality of connectors and plurality of receptacles can be made integral with stent (e.g., spot welded into position).

Further embodiments of the first stent-graft system include a plurality of sutures disposed around the plurality of connectors and plurality of receptacles. An angle of expansion of the connecting ring can be greater than about 90 degrees.

The term “connecting ring” refers to a structure (e.g., ring) adapted to connect to the first stent and to be disposed in the main graft body. The connecting ring can also be referred to as an annular ring. The connecting ring connects the first stent to the main graft body, provides for primary sealing and aids in forming a low profile stent-graft device wherein the first stent and main graft body are in a substantially end-to-end configuration.

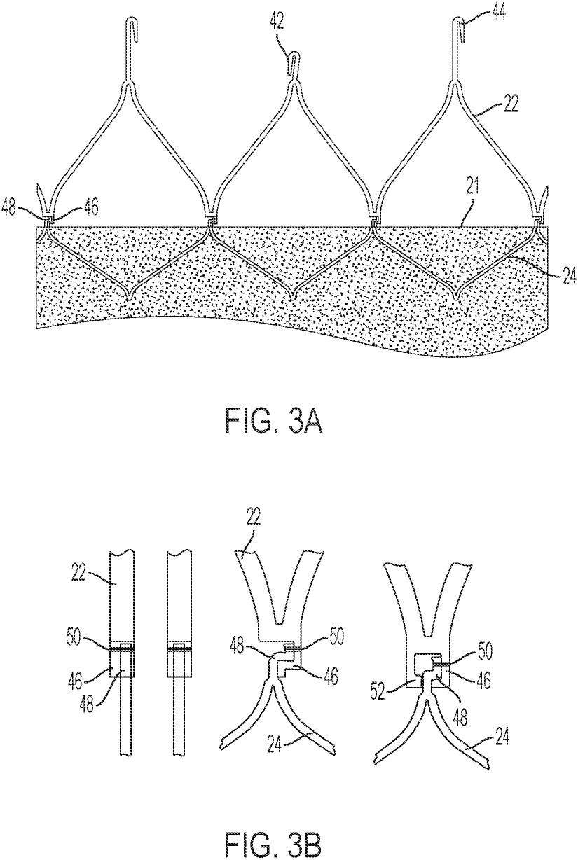

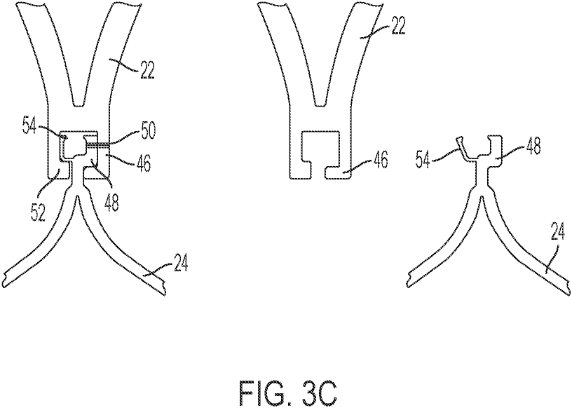

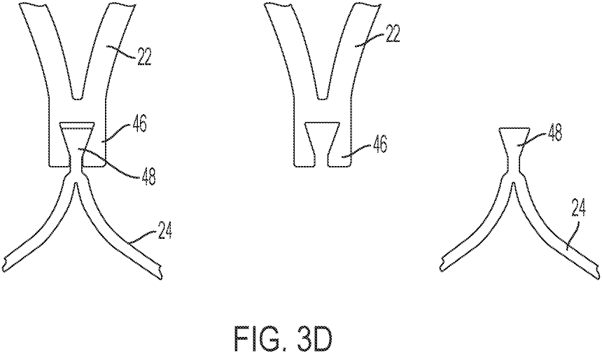

The term “connector” refers to, for example, a structure adapted to connect two components. For example, a connector (e.g., zig zag or other structure) disposed on a connecting ring can be adapted to fit into a hole on a receptacle disposed on a first stent as shown for example, in FIGS. 3A-3D.

It is to be understood that any suitable connector-receptacle system can be employed in order to bear a majority of the axial loading forces between the first stent and the connecting ring. For example, the connector can be shaped like a hook, half circle, triangle etc., and the receptacle can be a hole adapted to receive the hook, half circle, triangle, etc. Optional sutures can be provided to provide stability in place of or in addition to the exemplary connector-receptacle system, as needed.

The term “end-to-end configuration” refers to the connection of a component of the stent graft to another component at its margin (e.g., side by side, or end to end). An end-to-end configuration can avoid overlapping of components (i.e., coaxial) in the stent-graft introducer sheath. For example, a thick and strong top stent can be combined with an ePTFE graft or densified ePTFE graft. In one aspect, the components are arranged in series instead of coaxially. A coaxial configuration of components requires a larger diameter introducer sheath, a bigger hole in the blood vessel, and potentially a bigger vessel for introducing the stent-graft. As described herein, this exemplary configuration results in a smaller diameter stent-graft device that can be used, for example, in an artery located above the diaphragm and/or small arteries. In this aspect, the first stent can be thicker and more robust since it is not in an overlapping orientation with the main body graft.

The term “end-to-end configuration” with respect to the stent-graft systems and devices described herein, can also refer to a circumstance where the wall thickness of the connecting ring disposed in the main graft body is less than the wall thickness at a connection between the first stent and the graft or, in some instances, the connecting ring when the plurality of connectors receive the plurality of receptacles.

In some embodiments of the first stent-graft system, the connection between the plurality of connectors on the connecting ring and the plurality of receptacles on the first stent provide a stable configuration without increasing the overall diameter of the device. In addition, this configuration supports an angle of expansion of the connecting ring which is greater than about 90 degrees. For example, this configuration is very stable upon crimping in the delivery sheath, which allows for the stabilization of the connecting ring (due to its end-to-end attachment). If the connecting ring is not stabilized by the top stent, it would not be as stable upon crimping, and potentially cause damage and/or would not achieve the anticipated or desired crimp diameter.

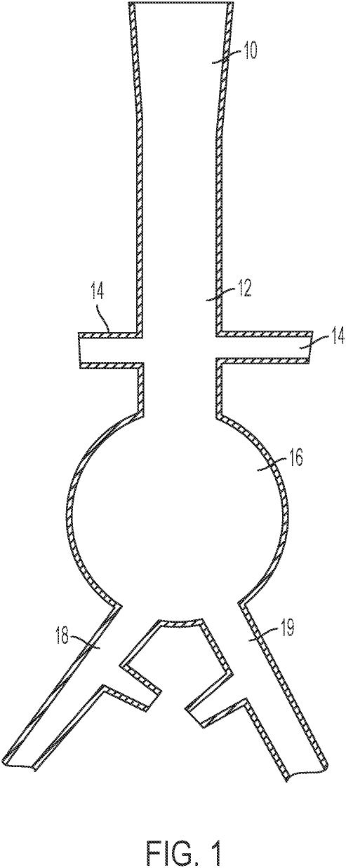

In another embodiment of the first stent-graft system, the main graft body bifurcates into two branches comprising a first limb gate and a second limb gate. In this aspect, the first limb gate and second limb gate can be adapted to be disposed in ipsilateral iliac artery 18 as shown, for example, in FIG. 1. In another aspect, the main graft branch does not bifurcate and can be configured to be positioned and deployed in a thoracic aorta or a blood vessel other than the aorta or an infrarenal aneurysm that does not extend to the iliac bifurcation or an aorto-uni-iliac tube endograft for placement in the aorta and one iliac artery.

An embodiment of the first stent-graft system further comprises at least a first iliac leg component. The stent-graft system can include at least a second iliac leg component. The iliac leg component can be adapted to be positioned in a constrained state through, for example, an insertion site blood vessel located above a diaphragm or small artery, through the main graft body, and through either the first limb gate or the second limb gate to either the ipsilateral or contralateral iliac artery where it can be deployed in an unconstrained state.

In some embodiments of the first stent-graft system, an overlap of the first iliac leg component with the first limb gate is not larger in diameter than an overlap of the second iliac component with the second limb gate.

Another embodiment of the first stent-graft system comprises barbs disposed on one or more of a first iliac leg component and a second iliac leg component for anchoring the one or more of the first iliac leg component and the second iliac leg component to a blood vessel.

In some embodiments of the first stent-graft system, the first iliac component and the second iliac component comprise a densified material (e.g., densified ePTFE).

Yet another embodiment of the first stent-graft system includes a second stent connected to a caudal (i.e., end closest to the tail or bottom of the body) end of the first limb gate and a third stent connected to a caudal end of the second limb gate. This embodiment can further comprise tethers disposed on a caudal end of each of the main graft body, the second stent, and the third stent for connecting to a caudad (i.e., toward the end or posterior of the body) positioning system.

The term “caudad positioning system” can refer to tools (e.g., catheter, guidewire, trigger or tether wire, etc.) that are configured to engage with the caudal end of the stent-graft system to allow an operator (i.e., doctor) to control and adjust the position of the stent-graft system in a target blood vessel.

Yet another embodiment of the first stent-graft system further comprises a plurality of sutures disposed around the plurality of connectors and plurality of receptacles. It is understood that the sutures can be placed in alternative or additional locations.

The first stent-graft system can further comprise barbs disposed on one or more of the first iliac leg component and the second iliac leg component for anchoring the one or more of the first iliac leg component and the second iliac leg component to a blood vessel.

The term “barbs” refer to sharpened projections configured to attach, associate, or anchor the stent-graft system in a blood vessel or components of the stent-graft system to each other. In this aspect, the plurality of barbs can function to prevent unintentional migration of the stent-graft device from a desired location. Barbs can be integral to the design (i.e., not made separately and then attached) or spot welded into position. The plurality of barbs can be configured to have alternating heights with respect to each other. In another aspect, each of the plurality of barbs can be oriented in a different direction with respect to each other.

In another embodiment of the first stent-graft system, the overlap of the first iliac leg component with the first limb gate and the overlap of the second iliac component with the second limb gate is not larger in diameter (e.g., oversized).

Optional tethers can be used, for example, to permit manipulation by a positioning system (e.g., guidewire, catheter, trigger wire) and aid in maneuvering the stent-graft device in the blood vessel and more precisely positioning the stent-graft system in a desired location. In this aspect, the tethers can be removably attached to the positioning system in order to push or pull the stent-graft system through the blood vessel.

The term “tether” refers to loop(s) or similar structures that can be used, for example, to removably attach to a portion of the positioning system and aid in positioning and deployment of the stent-graft device. A tether can be made from any suitable suture material or thin metallic wire and can be rigid or flexible.

In some embodiments of the first stent-graft device, the stent-graft system further comprises an optional annular element disposed inside the main graft body, and a plurality of connecting members associated with a plurality of locations on the annular element. In this aspect, the first stent has a first axial stent end and a second axial stent end, and the main body graft has a first axial graft end and a second axial graft end. The annular element can be made of any suitable material or can be integrated in the graft. The annular element can be continuous or discontinuous and can be disposed around the circumference of the graft or a portion of the circumference of the graft.

The annular element can be located at a position substantially adjacent to the first axial graft end. The plurality of connecting members can be configured to connect to the first axial stent end to maintain a substantially end-to-end axial connection between the first stent and the main graft body. The annular element can be made of any suitable material (e.g., graft material, biocompatible metal, ePTFE) and can be a discrete annular element that has different properties compared to the graft material (e.g., different density).

In one aspect, the connecting members can be attached to locations inside the main graft body without the need for an annular element. These locations can be arranged in any desired pattern including a circumferential manner around the inside of the main graft body.

The term “connecting members configured to connect” refers to a feature of a connecting member that engages with and is retained by a stent end in a substantially end-to-end arrangement. Examples of connecting members configured to connect to the first axial stent end are shown, for example, in FIGS. 3A, 3B, 3C, and 3D.

In some instances, the plurality of connectors project beyond the first axial graft end. In some instances, the plurality of connectors are circumferentially spaced apart. In a further aspect, the plurality of connectors are curved. In yet another aspect, each of the plurality of connectors can be different (e.g., length, curvature, material) from another connector.

In one aspect, the substantially end-to-end configuration comprises a gap between the first stent and the first axial graft end. In some instances, the gap is 0 to 2 mm. In other instances, the at least one of the plurality of connectors extends across the gap.

An embodiment of the first stent-graft device further comprises a centering device adapted to be disposed and expanded in a suprarenal aorta wherein the main graft body can be repositioned in an infrarenal aorta after the centering device is expanded.

The term “centering device” refers to a structure that can be removably or temporarily deployed and expanded in an artery or blood vessel (e.g., suprarenal aorta) to permit another device (e.g., stent-graft device) to be centered in another portion of the blood vessel with reference to the centering device.

In one aspect, the centering device can be a device with a plurality of arms that can be expanded radially wherein each arm can contact a wall of a blood vessel. In another aspect, the centering device can be a balloon wherein the balloon can be expanded and contact a wall of a blood vessel.

The centering device (e.g., basket, balloon) can be used, for example, to assist an operator (e.g., doctor) in positioning a stent-graft device in a target blood vessel by expanding the centering device such that it engages with a wall of the blood vessel. The operator can adjust the position of a second device (e.g., stent-graft device) with reference to a midpoint of the centering device when the centering device is expanded in the blood vessel.

The centering device can be use, for example, in a circumstance where there is a bend or angle in the anatomy of the blood vessel (e.g., suprarenal aorta angulation, see, e.g., Mathlouthi et al., Clinical research study Abdominal aortic and iliac artery aneurysms, Impact of suprarenal neck angulation on endovascular aneurysm repair outcomes, Journal of Vascular Surgery, Volume 71, ISSUE 6, P1900-1906, Jun. 01, 2020). In this circumstance, the centering device can be connected to the second device by a tether, suture, or other connection to further assist in centering the second device in reference to the centering device.

In some instances, the centering device can surround the central member for symmetrical expansion of the centering device or can be attached to only one point on the circumference of the central member to allow for eccentric expansion of the centering device. In this instance, the centering device is configured to push the central member away from one side of the aorta in order to counteract the effect of aortic angulation.

In some instances, the centering device is selected from the group consisting of a centering basket and a centering balloon. In some instances, the centering basket can be expanded, wherein at least a portion of the centering basket engages with a wall of the suprarenal aorta. In some instances, the centering balloon can be inflated, wherein at least a portion of the centering balloon engages with a wall of the suprarenal aorta.

Further aspects provide a first method, comprising repairing an abdominal aortic aneurysm in a patient, by puncturing an insertion site blood vessel above a diaphragm and creating a passage in the insertion site blood vessel. Next, a stent-graft system can be inserted in a passage of the insertion site blood vessel. The stent-graft system can comprise a main graft body, a first limb gate and a second limb gate. The main graft body of the stent graft system can be positioned and deployed in a target blood vessel (e.g., an infrarenal aorta, a juxtarenal aorta, a pararenal aorta, a thoracic aorta, or a suprarenal aorta) of a patient.

The first method described herein can be used, for example to insert straight tubular stent-grafts in an insertion site blood vessel located above the diaphragm. In some instances, insertion site blood vessels can include, for example, smaller blood vessels (i.e., blood vessels smaller in or equal to the diameter than a femoral artery of the same patient), arteries (e.g., common femoral artery, superficial femoral artery, popliteal artery, anterior tibial artery, posterior tibial artery, peroneal artery, axillary artery, and iliac artery) and veins (e.g., superior vena cava, inferior vena cava, femoral and popliteal veins, radial vein, cephalic vein, basilic vein, and axillary vein). In some instances, the insertion site blood vessel is the subclavian artery. In some instances, the methods described herein can be used to insert hemodialysis grafts and/or other man-made vascular grafts.

In some instances of the first method, the stent-graft system is adapted to be inserted through a single arterial puncture or incision located above a diaphragm of a subject or patient. Use of such a stent-graft system avoids the disadvantages of prior systems which require multiple arterial punctures or incisions. In some instances of the first method, the first stent and the main graft body are in a substantially end-to-end configuration

The insertion site blood vessel for the stent-graft systems described herein can be the second or proximal third portion of the axillary artery. This insertion site is the axillary artery proximal (e.g., closer to the heart) to the origin of the sub scapular artery. Use of this insertion site can, for example, avoid damaging branches of the brachial plexus.

In another instance of the first method, the femoral arteries are not used for insertion of the stent-graft device. Instead, the stent-graft device can be inserted through a single puncture or incision in an insertion site blood vessel located above the diaphragm (e.g., a “small artery” as described herein) that is not located in the leg. Such a procedure avoids the recovery time and discomfort associated with femoral artery punctures or incisions since the patient is able to walk right away.

In addition, as described above, the stent-graft device can be manipulated and positioned more easily from a “top-down” orientation in the aorta into both legs for repair of, for example, an abdominal aortic aneurysm. Therefore, the procedure takes less time, is less prone to error, and offers the patient a shorter recovery period with less pain.

In some instances of the first method, the single arterial puncture or incision is made in a small artery (e.g., an artery smaller or equal in diameter to a femoral artery from the same patient). The small artery can be located above the diaphragm. The artery can be selected from the group consisting of brachial, radial, ulnar, femoral, iliac, axillary, and carotid arteries. In yet another aspect, the artery is the brachial artery. In a further aspect, the artery is a femoral artery or the subclavian artery. In another instance, the artery is the second or proximal third portion of the axillary artery.

In yet another aspect, stent-graft systems can be deployed in the supra renal abdominal aorta to treat infrarenal abdominal aortic aneurysms. For example, side graft branches can be provided for the renal arteries, the celiac artery, and the superior mesenteric artery.

The term “positioned” refers to moving the stent-graft device (e.g., in a constrained or partially constrained configuration) through blood vessels to the desired location in a blood vessel (e.g., axial position) using any suitable mechanism (e.g., a positioning system). The positioning system can comprise a guide wire, a sheath over the guidewire, one or more catheters, a trigger wire, and other components capable of pushing, pulling, and deploying the stent-graft device. A doctor can visualize the progress of the positioning system and stent-graft device with an angiogram, fluoroscope, or other visualization system via radio-opaque markers located as appropriate.

In some instances of the first method, the main graft body stent-graft system can be positioned inferior to the lowest renal artery and in an orientation adapted to access the contralateral limb gate. Markers (e.g., radiopaque markers) can be used for orientation of the contralateral gate under fluoroscopic guidance. In another aspect, the device may be deployed above the renal arteries.

In some instances of the first method, the main graft body of the stent-graft system can be deployed in the infrarenal aorta, wherein the main graft body of the stent-graft system is in a substantially blood-tight seal with respect to a wall of the infrarenal aorta. The term “deployed” or “deploying” refers to transforming the stent-graft device from a constrained to an unconstrained or open configuration where the stent-graft device can treat the aneurysm or other condition.

The stent-graft system can also include a first iliac leg component and a second iliac leg component. The first iliac leg component can be positioned and deployed in a first branch of the iliac artery. Next, the second iliac leg component can be positioned and deployed in a second branch of the iliac artery. In this aspect, the first and second iliac leg components can be in a substantially blood-tight seal with respect to the main graft body (e.g., via the graft branches).

The first iliac leg component can be in a substantially blood-tight seal with respect to the first branch of the iliac artery. The second iliac leg component can also be in a substantially blood-tight seal with respect to the second branch of the iliac artery.

The term “substantially blood-tight seal” refers to limiting (e.g., by 95, 90, 85, or 80%) or eliminating an endoleak (e.g., a blood leak back into an aneurysm sac following an EVAR procedure).

In another instance of the first method, the main graft body is positioned in the target blood vessel (e.g., an infrarenal aorta, a juxtarenal aorta, a pararenal aorta, a thoracic aorta, or a suprarenal aorta) in a constrained state using a guidewire. The main graft body can be further positioned in the target blood vessel (e.g., an infrarenal aorta, a juxtarenal aorta, a pararenal aorta, a thoracic aorta, or a suprarenal aorta) by partially unsheathing the main graft body. In one aspect, the main graft body is unconstrained after the main graft body is deployed in the infrarenal aorta.

In some instances of the first method, a sealing stent can be deployed in the main graft body. The term “sealing stent” refers to a stent configured to limit or prevent an endoleak.

The first iliac leg component can be positioned in the first branch of the iliac artery in a constrained state through the main graft body using a guidewire. The first iliac leg component can also be further positioned in the first branch of the iliac artery by partially unsheathing the first iliac leg component. In this aspect, the first iliac leg component is unconstrained after the first iliac leg component is, for example, fully unsheathed and deployed in the first branch of the iliac artery.

The second iliac leg component can be positioned in the second branch of the iliac artery in a constrained state through the main graft body using a guidewire. The second iliac leg component can also be further positioned in the second branch of the iliac artery by partially unsheathing the second iliac leg component. In this aspect, the second iliac leg component is unconstrained after the second iliac leg component is, for example, fully unsheathed and deployed in the second branch of the iliac artery.

In some instances of the first method, the stent-graft system further comprises a first stent and a connecting ring. The first stent can have a plurality of receptacles and the connecting ring can have a plurality of connectors adapted to receive the plurality of receptacles. In this aspect, the connecting ring can be disposed in the main graft body, and the first stent and the main graft body are in a substantially end-to-end configuration.

In some instances of the first method, an optional annular element is disposed inside the main graft body, and a plurality of connecting members is associated with a plurality of locations on the annular element. In this aspect, the first stent has a first axial stent end and a second axial stent end, and the main body graft had a first axial graft end and a second axial graft end. The annular element can be located at a position substantially adjacent to the first axial graft end. The annular element can be made of any suitable material or can be integrated in the graft. The annular element can be continuous or discontinuous and can be disposed around the circumference of the graft or a portion of the circumference of the graft.

The annular element can be located at a position substantially adjacent to the first axial graft end. The plurality of connecting members can be configured to connect to the first axial stent end to maintain a substantially end-to-end axial connection between the first stent and the main graft body. The annular element can be made of any suitable material (e.g., graft material, biocompatible metal, ePTFE) and can be a discrete annular element that has different properties compared to the graft material (e.g., different density).

In further instances of the first method, the connecting members can be attached to locations inside the main graft body without the need for an annular element. These locations can be arranged in any desired pattern including a circumferential manner around the inside of the main graft body.

The connecting members can be configured to connect to the first axial stent end to maintain a substantially end-to-end axial connection between the first stent and the main graft body.

In some instances of the first method, the plurality of connectors project beyond the first axial graft end. In some instances, the plurality of connectors and the plurality of receptacles are circumferentially spaced apart.

The substantially end-to-end configuration can include a gap (e.g., from greater than 0 to 2 mm) between the first stent and the graft. In some instances, the plurality of connectors extend across the gap.

Further aspects provide a second stent-graft system having an endograft deployment system for deploying an endograft in a target blood vessel (e.g., an infrarenal aorta, a juxtarenal aorta, a pararenal aorta, a thoracic aorta, or a suprarenal aorta) of a patient or subject. The endograft deployment system comprises an outer tube having a central inner member comprising an endograft and a carrier tube comprising a tether wire having a caudad end and a more cephalad portion, a top stent surrounding the central inner member, wherein the top stent comprises a plurality of hooks having a plurality of receptacles, and a plurality of sutures, wherein a first end of at least a first suture is disposed through one of the plurality of receptacles for retaining the top stent in a constrained configuration, and a second end of the first suture is affixed to the more cephalad portion of the tether wire. The first end of the at least a first suture can be attached to, for example, the tether wire. Movement of the tether wire can control removal of the plurality of sutures from the plurality of receptacles, release the top stent from a constrained to an unconstrained configuration, and remove the plurality of sutures from the blood vessel of the subject. In some instances, the tether wire may be removed while the sutures may be left in the patient.

Aspects described herein provide stent-graft systems and methods to substantially remove sutures from the blood vessel of a subject after deployment of a stent-graft system. In some instances, the first end of the at least a first suture is formed into a loop, and the loop is disposed around the caudad end of the tether wire.

Sutures can be an important or essential component for many surgical procedures, including EVAR. However, the presence of a suture or a portion of a suture after a cardiovascular surgical procedure can cause serious side effects, including embolism, negative impact on blood vessel healing, and injury to coronary arteries. See, e.g., Lee et al., Suture knot embolism—a rare complication of percutaneous arterial closure device, Cardiovascular Pathology Volume 19, Issue 1, January-February 2010, Pages 63-64; Osama Hazim Al Hayini, Effect of different suture materials on healing of blood vessels in dogs, Iraqi Journal of Veterinary Sciences 26:77-82 (January 2012); Annuloplasty Rings 510(k) Submissions - Final Guidance for Industry and FDA (Food and Drug Administration) Staff, US FDA Guidance January 31, 2001.

In some instances, the endograft deployment system further comprises a shelf disposed below the first end of the at least a first suture for initially retaining the first end of the at least a first suture above the shelf. In some instances, the shelf further comprises a first opening for receiving the central inner member. The endograft deployment system can comprise a second opening for receiving the carrier tube. The endograft deployment system can comprise a third opening for receiving at least one of the plurality of sutures. In some instances, the third opening comprises three partial openings.

Aspects described herein provide a second method, comprising puncturing a femoral artery and creating a passage in the femoral artery, inserting a stent-graft in the passage of the femoral artery with a stent-graft deployment system having an outer tube comprising a central inner member comprising an endograft and a carrier tube comprising a tether wire.

In this aspect, the tether wire can have a caudad end and a more cephalad portion. The stent-graft deployment system can further comprise a top stent surrounding the central inner member, wherein the top stent comprises a plurality of hooks having a plurality of receptacles; and a plurality of sutures. In this aspect a first end of at least a first suture is disposed through one of the plurality of receptacles for retaining the top stent in a constrained configuration, and a second end of the first suture is affixed to the more cephalad portion of the tether wire. The first end of the at least a first suture can be attached to, for example, the tether wire.

The tether wire can be moved in order to remove the plurality of sutures from the plurality of receptacles, release the top stent from a constrained to an unconstrained configuration, and remove the plurality of sutures from the blood vessel of the subject.

The first end of the at least a first suture can be formed into a loop, and the loop can be disposed around the caudad end of the tether wire

The first limb gate of the stent-graft device can be positioned and deployed in a first branch of the iliac artery wherein the first limb gate of the stent-graft device is in a substantially blood-tight seal with respect to a wall of the first branch of the iliac artery and the main graft body.

The second limb gate of the stent-graft device can be positioned and deployed in a second branch of the iliac artery. As described herein, the positioning and deployment of the second limb gate in the second branch of the iliac artery can be accomplished without the need for a second puncture or incision in the corresponding femoral artery. In this aspect, the stent-graft device can be in a substantially blood-tight seal with respect to a wall of the second branch of the iliac artery.

Aspects described herein also provide alternative methods of repairing an abdominal aortic aneurysm in a patient by puncturing or making an incision in the femoral arteries and creating a passage in the femoral arteries. In this aspect, a stent-graft system (e.g., a main graft body, a first limb gate and a second limb gate) can be inserted in the passage of the femoral artery. The main graft body of the stent-graft system can be positioned and deployed in an infrarenal aorta of the patient.

The main graft body of the stent-graft system can be in a substantially blood-tight seal with respect to a wall of the infrarenal aorta. In this aspect, the stent-graft system can have a first iliac leg component and a second iliac leg component. The first iliac leg component can be positioned and deployed in a first branch of the iliac artery and the second iliac leg component can be positioned in a second branch of the iliac artery and the main body graft.

In this aspect, the first iliac leg component can be in a substantially blood-tight seal with respect to the first branch of the iliac artery and the second iliac leg component can be in a substantially blood-tight seal with respect to the second branch of the iliac artery

In another aspect, the contralateral limb gate (i.e., outlet of second limb gate) can be cannulated from a “top down” approach using access from an insertion site blood vessel located above a diaphragm of a patient or subject (e.g., axillary artery) using a maneuverable guidewire and steerable catheter. Alternatively, a guidewire with a bend near the nose cone may be used instead of a catheter and guidewire in combination. In some instances, a retrograde femoral artery approach can be used.

In another aspect, the main graft body is positioned in the infrarenal aorta in a constrained state using a guidewire. In another aspect, the main graft body is further positioned in the infrarenal aorta by partially unsheathing the main graft body. In yet another aspect, the main graft body is unconstrained after the main graft body is deployed in the infrarenal aorta.

Aspects provide a further alternative method of repairing an abdominal aortic aneurysm in a patient by puncturing the femoral arteries and creating a passage in the femoral arteries. The stent-graft device can be inserted in a passage of the femoral artery. In this aspect, the stent-graft device can comprise a main graft body, a first limb gate and a second limb gate. The main graft body of the stent-graft device can be positioned and deployed in a target blood vessel (e.g., an infrarenal aorta, a juxtarenal aorta, a pararenal aorta, a thoracic aorta, or a suprarenal aorta) of the patient. In this aspect, the main graft body of the stent-graft device is in a substantially blood-tight seal with respect to a wall of the infrarenal aorta. The main graft body of the stent-graft device can have a substantially blood-tight seal with respect to a wall of the target blood vessel. In another aspect, only one femoral artery is punctured.

Aspects described herein provide a third stent-graft system for repair of an aneurysm in a target blood vessel having a main graft body comprising a sealing stent at least partially disposed in the main graft body. In this aspect, the stent-graft system can be configured to be inserted through a single arterial puncture or incision in an insertion site blood vessel located above a diaphragm of a patient. In some instances, the diameter of the stent-graft system in a constrained configuration is adapted to be inserted into a small artery (e.g., an artery smaller or equal in diameter to a femoral artery from the same patient). In some instances, the diameter of the stent-graft system in a constrained configuration is from about 13 to about 22 French or 6 to about 13 French.

In one aspect, the sealing stent has a plurality of struts. The plurality of struts can have a strut width of about 0.013 to 0.016 inches. In some instances, the sealing stent has a plurality of struts and the plurality of struts have a strut wall thickness of about 0.016 to about 0.020 inches.

In some instances, the main graft body has a first axial end and a second axial end. The sealing stent can be disposed adjacent to the first axial end. The sealing stent can further comprise a plurality of hooks.

In some instances, the plurality of hooks extend beyond the first axial end of the main graft body. In some instances, each of the plurality of hooks further comprises a radiopaque marker.

In further aspects, each of the plurality of hooks can be oriented in a right-hand orientation or a left-hand orientation. In yet another aspect, an orientation of each of the plurality of hooks is different from an adjacent hook. In some instances, the plurality of hooks are adapted to be reversibly retained by a looped wire for repositioning of the main graft body in the infrarenal aorta.

In some instances, the looped wire is inserted into the target blood vessel through a recapture sheath. The recapture sheath can be from about 2 to 4 French. In one aspect, recapture of the stent-graft device and repositioning of the stent-graft device can only occur from an insertion site blood vessel located above a diaphragm of a patient or subject.

The plurality of hooks can further comprise a plurality of hook eyelets. The looped wire can be removably inserted through at least one of the plurality of hook eyelets.

In some instances, an entire stent-graft device (e.g., endograft) can be recaptured in a situation where the procedure needs to be aborted using, for example, a 10 French sheath. In another instance, the sheath can be exchanged for a higher size sheath during the procedure to recapture the entire stent-graft device if it is necessary to abort the entire procedure.

Aspects described herein provide alternative methods of repairing an abdominal aortic aneurysm in a patient by (a) inserting a first guidewire into a small artery with a first access profile of about 3 French; (b) inserting a catheter into an ipsilateral iliac artery with a second access profile of about 5 French; (c) deploying a main graft body from a main graft body deployment system in an infrarenal aorta of the patient with a third access profile of about 10 French; (d) removing an inner component of the main graft body deployment system and leaving a first sheath and the first guidewire in the infrarenal aorta; (e) deploying a first limb gate in ipsilateral iliac artery using the first sheath and the first guidewire; (f) moving the first guidewire from the ipsilateral iliac artery to a contralateral iliac artery; and (g) deploying a second limb gate in the contralateral iliac artery using the first sheath and first guidewire.

Further aspects provide alternative methods of repairing an abdominal aortic aneurysm in a patient by (a) inserting a first guidewire into a small artery into an ipsilateral iliac artery of a patient; (b) insert a main body graft and a first sheath over the first guidewire into the infrarenal aorta; (c) deploying a main body graft in the infrarenal aorta of the patient; (d) inserting a first limb gate into an ipsilateral iliac artery of the patient and deploying the first limb gate; (e) removing the first guidewire from the ipsilateral artery; (f) inserting a 4-5 French directional catheter through the first sheath; (g) cannulating a contralateral iliac gate and a contralateral iliac artery with the first guidewire; (h) inserting a second limb gate into the contralateral iliac artery; and (i) deploying the second limb gate into the contralateral iliac artery.

In some instances, the methods further comprise deploying one or more sealing stents in the infrarenal aorta. The sealing stents can prevent or minimize endoleaks.

Aspects described herein provide a third method, comprising positioning a main graft body of a stent-graft system in an infrarenal aorta of a subject by advancing a main graft body delivery system to a target location, the main graft body delivery system comprising the main body graft and a centering device. Next, the main body graft can be positioned in the infrarenal aorta in a first position at the target location and it can be determined if the first position of the main graft body is centered in the infrarenal aorta at the target location. The centering device can be deployed in a suprarenal aorta in a centered position if the first position of the main graft body is not centered in the infrarenal aorta at the target. The main graft body can be repositioned in the infrarenal aorta in a second position determined from the centered position if the first position of the main graft body is not centered in the infrarenal aorta at the target.

In some instances of the third method, the insertion site blood vessel for the stent-graft device is located above a diaphragm of the patient.

In some instances of the third method, the centering device is selected from the group consisting of a centering basket and a centering balloon. In some instances, the centering basket can be expanded, wherein at least a portion of the centering basket engages with a wall of the suprarenal aorta. In some instances, the centering balloon can be inflated, wherein at least a portion of the centering balloon engages with a wall of the suprarenal aorta. In some instances, the outward force exerted by a centering basket or the outer surface of a centering balloon is greater than a longitudinal force exerted by a sheath or other delivery device when conforming to the curvature of a blood vessel.

In some instances of the third method, the main graft body delivery system further comprises a sheath comprising the main graft body, wherein retracting the sheath exposes the main graft body in the infrarenal aorta prior to positioning the main graft body in the infrarenal aorta.

In some instances of the third method, the sheath further comprises the centering device, wherein further retraction of the sheath exposes the centering device in the suprarenal aorta prior to deploying the centering device.

In some instances of the third method, the main graft body delivery system further comprises a main graft body restraining device and a main graft body releasing device. The main graft body restraining device can maintain the main graft body cephalad end in a constrained configuration. The main graft body releasing device can induce release of the main graft body into an unconstrained configuration.

For example, the restraining device can comprise a main graft body restraining wire adapted to control the position of the main graft body. The term “adapted to control” refers to, for example, the ability of an operator (e.g., a doctor) to move the restraining wire in a manner that alters the position of the main graft body in an artery (e.g., moving the main graft body in a caudad or cephalad direction).

In some instances of the third method, the main graft body releasing device comprises a snare loop adapted to release the main graft in the infrarenal aorta. The term “snare loop” refers to a material (e.g., wire, suture) configured in a loop or circular shape where the diameter of the loop can be reduced around a target. See, e.g., U.S. Pat. 8,628,540. In the context of surgery, the loop can be disposed around or through a target and closed in order to remove or manipulate a target (e.g., snare loop disposed through eyelets attached to a stent).

In some instances, the snare loop can re-constrain the cephalad end of the main graft body after an expansion of the cephalad end of the main graft body and control repositioning of the axial position of the main graft body in the infrarenal aorta. In this aspect, the main graft body can be positioned and repositioned in the infrarenal aorta by the operator. In one aspect, the snare loop can be removed from the subject. In another aspect, the caudad end of the main graft body can be secured by a trigger wire to the delivery system shaft.

In a further instance of the third method, the main graft body has a caudad end and a cephalad end, and a location of the first position is determined by a location of markers on the cephalad end (e.g., radiopaque markers) of the main graft body. In some instances, the location of the markers is caudad to the renal arteries. In some instances, the method further includes securing the caudad end of the main graft body to the main graft body restraining device with a trigger wire.

In some instances, repositioning of the stent-graft device can only occur from an insertion site blood vessel located above a diaphragm of a patient or subject.

Aspects described herein provide a fourth stent-graft system for repairing an aneurysm in a target blood vessel (e.g., an infrarenal aorta, a juxtarenal aorta, a pararenal aorta, a thoracic aorta, or a suprarenal aorta) having a top stent having a plurality of positioning receptacles; a main graft body wherein the top stent and the main graft body are in a substantially end-to-end configuration and wherein the top stent and the main graft body are disposed around an inner member; a snare tube comprising a snare loop, wherein a first end of the snare loop is disposed in the snare tube, and a second end of the snare loop is disposed from the snare tube, through the positioning receptacles, around the inner member, and into the snare tube; wherein the snare tube is parallel to the inner member and first end of the snare loop is adjacent to the second end of the snare loop.

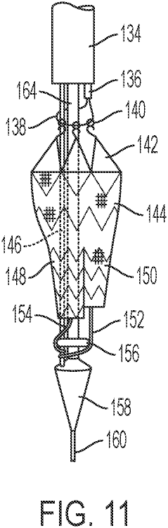

In one embodiment of the fourth stent-graft system, the stent-graft system can optionally be configured to be inserted through a single arterial puncture or incision in an insertion site blood vessel located above a diaphragm of the patient. An example of this stent-graft system is illustrated in FIG. 11.

In another embodiment of the fourth stent-graft system, each of the plurality of positioning receptacles further comprises an eyelet and the second end of the snare loop is disposed through at least one eyelet. In some instances, the stent-graft system further comprises an outer sheath disposed around the inner member.

The insertion site blood vessel can have a diameter less than or equal to the diameter of a femoral artery of the patient. The diameter of the stent-graft system in a constrained configuration can be from about 13 to about 22 French or about 6 to about 13 French. The main graft body can comprise a densified material.

In an embodiment of the fourth stent-graft system, the main graft body bifurcates and further comprises a first limb gate and a second limb gate. The first limb gate can comprise a first iliac leg component. The second limb gate can comprise a second iliac leg component. The top stent and the main graft body can be in a substantially end-to-end configuration.

In a further embodiment of the fourth stent-graft system, the stent-graft system further comprises a trigger or tether wire for positioning the first limb gate and second limb gate. The positioning system can further comprise a first limb gate tether and a second limb gate tether. The first limb gate tether and second limb gate tether can be retained by the trigger wire.

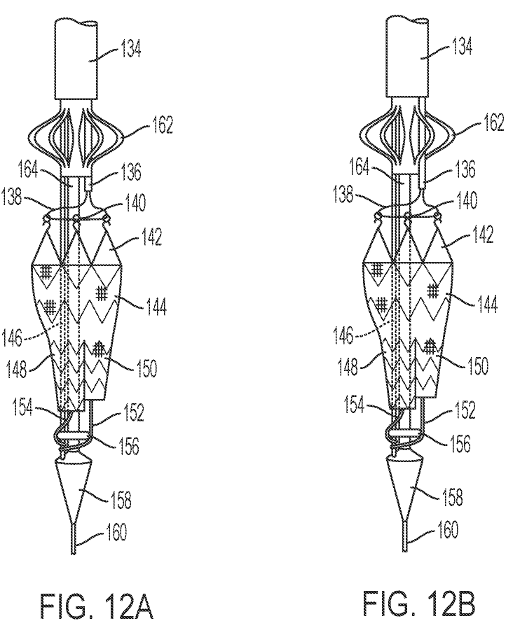

The fourth stent-graft system can further comprise a centering device for centering the main graft body in the infrarenal aorta as illustrated, for example, in FIG. 12A.

The main graft body, top stent, and centering device can initially be contained within an outer sheath. When the outer sheath is removed from the main graft body and the top stent, the top stent can be deployed, and the centering device can be pushed axially in a caudad direction to the aortic neck (e.g., non-dilated region above the aneurysm) and into the main graft body. The centering device can be deployed inside the graft in the portion of the main graft body nearest to the heart. The centering device can then be retracted, and the snare loop can be released to deploy the top stent.

Alternative aspects provide methods of positioning the stent-graft system in a target blood vessel described herein (e.g., FIG. 11), by shortening the length of the snare loop wherein the top stent is collapsed from a deployed configuration to a constrained configuration and the top stent is moved into the outer sheath; adjusting the location of the stent-graft system in the infrarenal aorta; and lengthening the snare loop wherein the top stent is moved out of the outer sheath and the top stent is expanded from a constrained configuration to a deployed configuration in the target blood vessel. In some instances, repositioning of the stent-graft device can only occur from an insertion site blood vessel located above a diaphragm of a patient or subject.

In circumstances where high suprarenal angulation leads to a sub optimal deployment (e.g., angles that start at the renal arteries) the snare loop can be opened, and the centering device can be moved in a caudad direction into the infrarenal aorta to the most cephalad portion of the graft. The snare loop can be closed to reconstrain the hooks permitting the centering device to center the endograft in the infrarenal aorta. The snare loop can be opened in order to retract the centering device. The centering device can help to uniformly open the upper end of the endograft.

In some instances (e.g., FIG. 12A), snare loop and snare tube are disposed outside the perimeter of the centering device and inside the perimeter of the outer sheath. In this example, when centering basket is pushed down, it will pass through the center of the snare loop, top stent, and into the inner member.

Aspects described herein provide a fifth stent-graft system for repair of an aneurysm in a target blood vessel (e.g., an infrarenal aorta, a juxtarenal aorta, a pararenal aorta, a thoracic aorta, or a suprarenal aorta) having a top stent comprising a plurality of positioning receptacles; a main graft body wherein the top stent and the main graft body are in a substantially end-to-end configuration and wherein the top stent and the main graft body are disposed around an inner member; a snare loop is disposed through the positioning receptacles with a degree of rotation around the top stent of greater than 360 degrees (e.g., 300 to 800 degrees, 540 degrees) wherein a first end of the snare loop is disposed substantially symmetrically (e.g., concentrically) with respect to the inner member. In one embodiment of the fifth stent-graft system, the stent-graft system can be configured to be inserted through a single arterial puncture or incision in an insertion site blood vessel located above a diaphragm of the patient.

In a further embodiment of the fifth stent-graft system, each of the plurality of positioning receptacles further comprises an eyelet and the second end of the snare loop is disposed through at least one eyelet. The stent-graft system can comprise an outer sheath disposed around the inner member. The insertion site blood vessel can have a diameter less than or equal to the diameter of a femoral artery of the patient. The diameter of the stent-graft system in a constrained configuration can be from about 13 to about 22 French or about 6 to about 13 French. The main graft body can comprise a densified material.

In one embodiment of the fifth stent-graft system, the main graft body bifurcates and further comprises a first limb gate and a second limb gate. The first limb gate can comprise a first iliac leg component. The second limb gate can comprise a second iliac leg component.

In some embodiments of the fifth stent-graft system, the top stent and the main graft body are in a substantially end-to-end configuration. The stent-graft system can further comprise a trigger or tether wire for positioning the first limb gate and second limb gate. The stent-graft system can further comprise a first limb gate tether and a second limb gate tether. The first limb gate tether and second limb gate tether can be retained by the trigger wire.

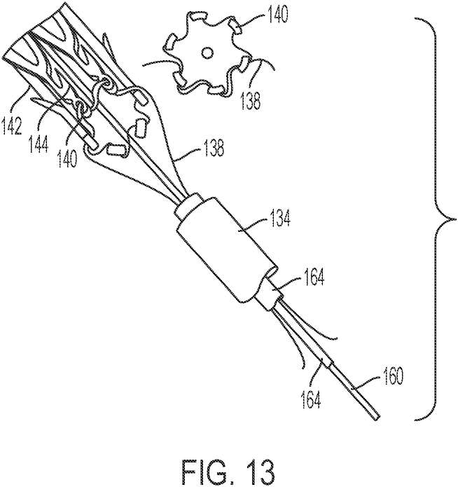

Aspects described herein provide methods of positioning the stent-graft system in a target blood vessel described herein (e.g., FIG. 13) by shortening the length of the snare loop wherein the top stent is collapsed from a deployed configuration to a constrained configuration and the top stent is moved into the outer sheath; adjusting the location of the stent-graft system in the target blood vessel; and lengthening the snare loop wherein the top stent is moved out of the outer sheath and the top stent is expanded from a constrained configuration to a deployed configuration in the target blood vessel. In this aspect, repositioning of the stent-graft device can be performed from an insertion site blood vessel located above a diaphragm of a patient or subject.

FIG. 1 shows the anatomy of a portion of the aorta including descending thoracic aorta 10, suprarenal abdominal aorta 12, renal arteries 14, aneurysmal infrarenal aorta 16, and the ipsilateral iliac artery 18 and contralateral iliac artery 19.

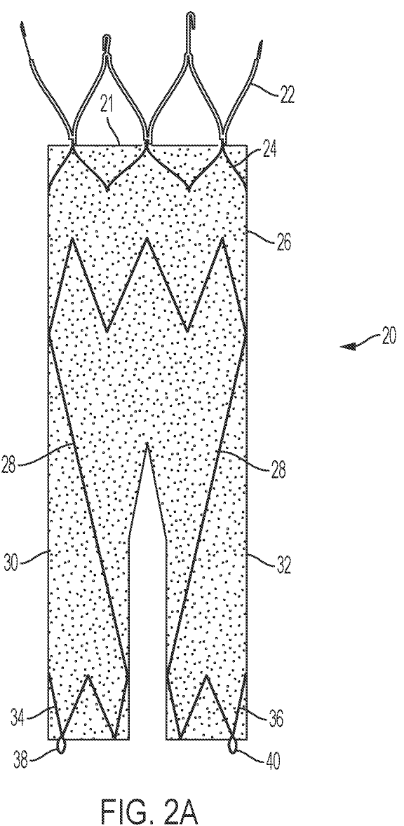

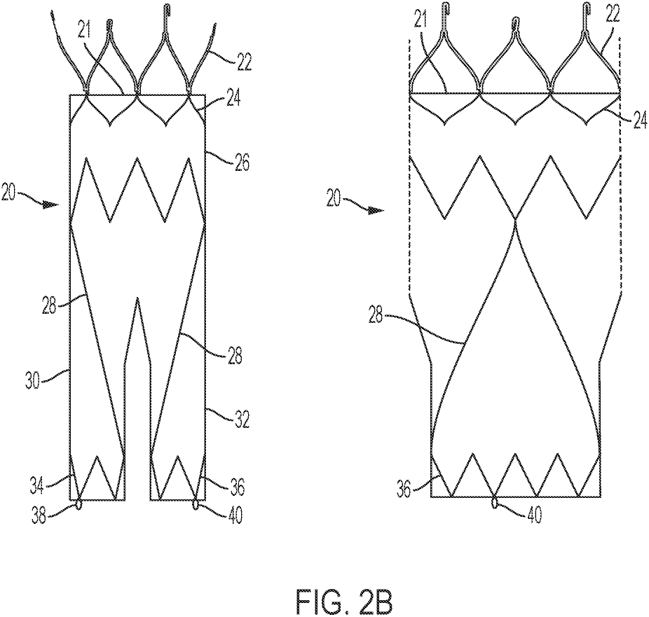

FIG. 2A shows an exemplary stent-graft device 20 in accordance with some instances of the first stent-graft system described herein. In this example, stent-graft device 20 includes graft 21, first stent 22, connecting ring 24, main graft body 26, connecting bar 28, first limb gate 30, second limb gate 32, second stent 34, third stent 36, first tether 38, and second tether 40. Connecting ring 24 and stents (22, 34 and 36) can be fully encapsulated in graft body material 26, or partially encapsulated in graft body material 26 or not encapsulated in graft body material 26 at all.

FIG. 2B shows a flattened side view of the exemplary stent-graft device of FIG. 2A in accordance with aspects described herein.

FIG. 3A shows an exemplary portion of first stent 22 having lower barb 42 and higher barb 44 and receptacle 46. Connector 48 is shown on connecting ring 24 embedded in graft 21.

FIG. 3B shows close up front and side views of receptacle 46 and connector 48 with an angled zig-zag configuration of receptacle 46 adapted to engage with and fit into connector 48. Optional sutures 50 are shown wrapped around connector 48 inside receptacle 46. Optional receptacle stabilizer 52 is shown adjacent to connector 48 to provide additional axial support.

FIG. 3C shows close up front views of an alternate configuration of a portion of first stent 22, a portion of connecting ring 24, receptacle 46, connector 48, sutures 50, and receptacle stabilizer 52 with optional connector stabilizer 54 adapted to fit receptacle stabilizer 52.

FIG. 3D shows close up front views of an alternate configuration of a portion of first stent 22, a portion of connecting ring 24, receptacle 46, and connector 48 where connector 48 has a triangular shape adapted to fit into a triangular shaped receptacle 46.

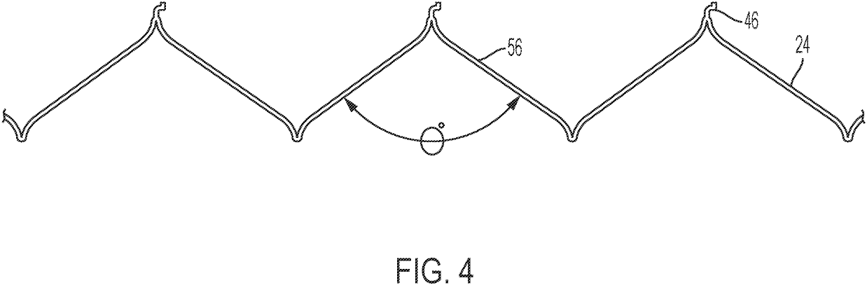

FIG. 4 shows an alternative aspect where arms 56 of the connecting ring or first stent 24 are at angle of 110 degrees with respect to each other. Conventional stent-graft devices can be unstable if the angles between arms of the connecting ring or stent are greater than 60 degrees.

Prior stent-graft devices in an end-to-end configuration using angles between stent arms greater than about 60 degrees are susceptible to instability. FIG. 4 shows connecting ring 24 where adjacent arms 56 are at an angle greater than 90 degrees. Stand-alone z stents are typically unstable at angles greater than 60 degrees. In this aspect, the attachment of the connecting ring to a robust, stable first stent (e.g., such as first stent 22 seen in FIG. 3A) via the connectors/receptacles provides for an increased angle for the connecting ring. The increased angle provides for a shortened stent length and hence a shorter seal length. Without be bound by theory, it is believed that the seal length is coupled with the length from the cranial edge of the graft until the first full ring of stent apposition. Short neck aneurysms are not able to be treated based on some designs which have long stents in the seal zone. In some instances, the connecting ring and stents can be encapsulated in the graft, are not encapsulated in the graft, or can be partially encapsulated in the graft.

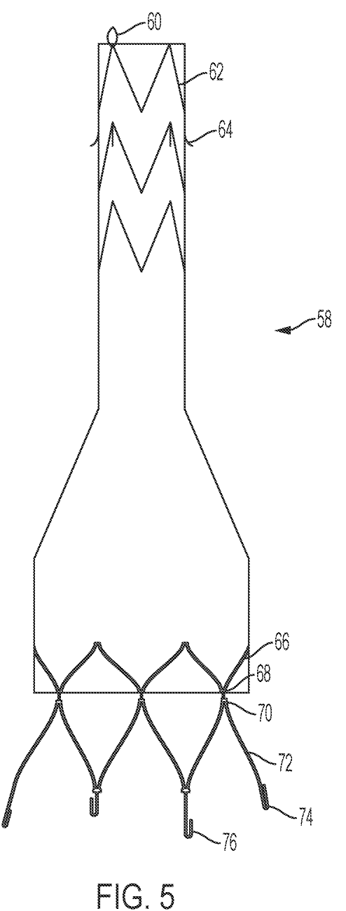

FIG. 5 shows an exemplary configuration of iliac leg component 58 having leg component tether 60, leg component top stent 62, leg component modular junction barb 64, leg component connecting ring 66, leg component connector 68, leg component receptacle 70 on leg component outer stent 72 having lower barb 74 and upper barb 76. Iliac leg component 58 can be maneuvered and positioned using leg component tether 60.

Deployment of iliac leg component 58 expands leg component top stent 62 and leg component outer stent 72 as iliac leg component 58 is moved from a constrained to an unconstrained configuration. In an unconstrained configuration, iliac leg component 58 can form a substantially blood tight seal against the iliac artery. Leg component barbs 64 can provide further stability with respect to positioning iliac leg component 58 within the main body branch. Leg component connecting ring 66 can be configured similarly to connecting ring 24 including having leg component 68 adapted to fit into leg component receptacle 70 on leg component outer stent 72. Iliac leg component 58 can be deployed in the contralateral iliac artery, the ipsilateral iliac artery, or both depending on the needs of the patient.

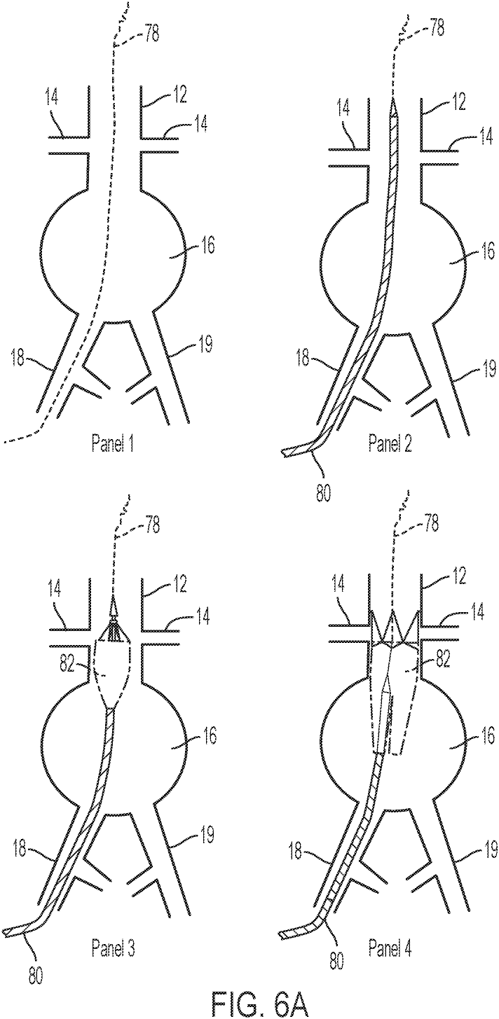

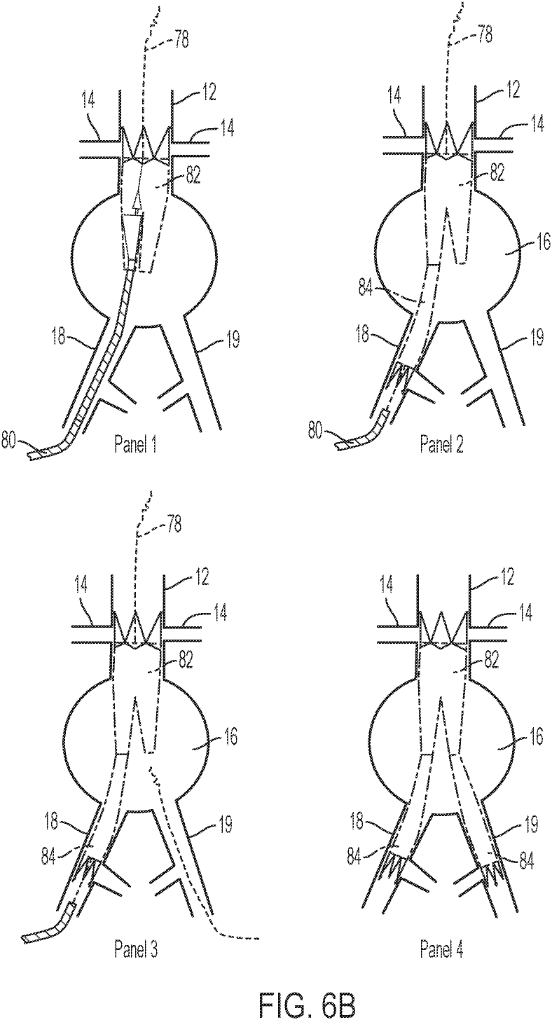

FIG. 6A shows an example of positioning and deploying a contemporary (currently typical) main graft body using the typical EVAR method of repairing an abdominal aortic aneurysm. In Panel 1, guidewire 78 is inserted through a femoral puncture or incision into iliac artery 18, aneurysmal infrarenal aorta 16, past renal arteries 14, into suprarenal abdominal aorta 12 in a “bottom up” approach. In Panel 2, delivery system sheath 80 containing a main graft body 82 is inserted over guidewire 78 and tracked over the guidewire into the suprarenal abdominal aorta 12. In Panel 3, the main graft body 82 is positioned, partially unsheathed, and can have its position adjusted before final release. In Panel 4, main body 82 is fully unsheathed and deployed in infrarenal abdominal aorta 12 as shown just below the renal arteries 14.

FIG. 6B shows an example of positioning and deploying typical currently available iliac leg components using the currently typical EVAR method of repairing an abdominal aortic aneurysm. The delivery system sheath 80 is left in place. As shown in Panel 1, leg component 84 is inserted into the delivery system sheath 80 and through the femoral artery puncture or incision in a “bottom up” approach over the guidewire 78 into a graft branch of main graft body 82. Iliac leg component sheath 84 is positioned and partially retracted exposing and allowing the iliac leg component to expand in the overlap zone. In Panel 2, leg component 84 is fully unsheathed and deployed into one leg of iliac artery 18.

In Panel 3, guidewire 78 is inserted into a second femoral puncture or incision in a “bottom up” approach to position it into another leg branch of main graft body 82, referred to as cannulation. Accurate positioning of leg component 84 by cannulation in a “bottom up approach” can be very difficult. In Panel 4, once cannulation is achieved with the guidewire, leg component 84 is positioned and deployed in the other leg of iliac artery 18.

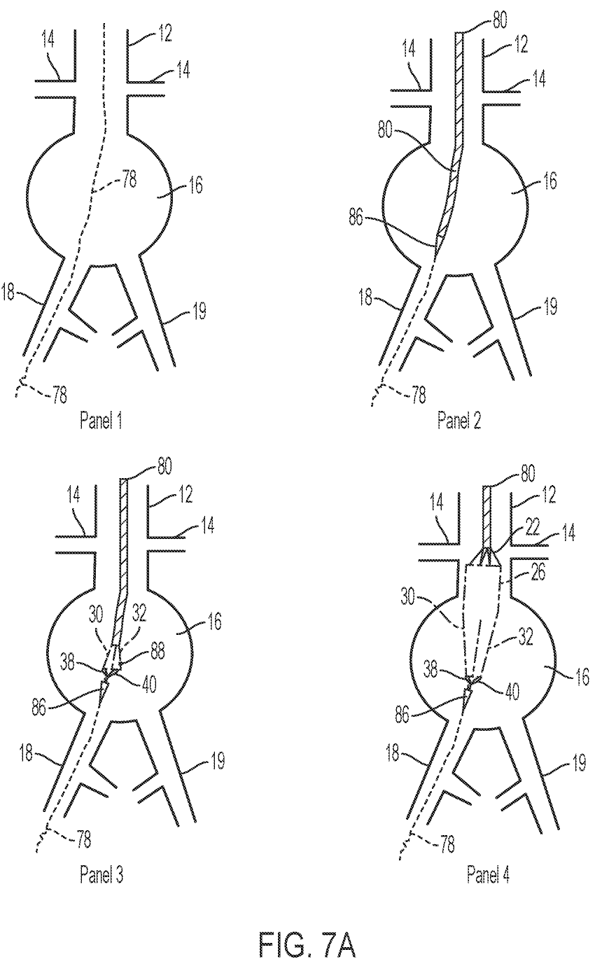

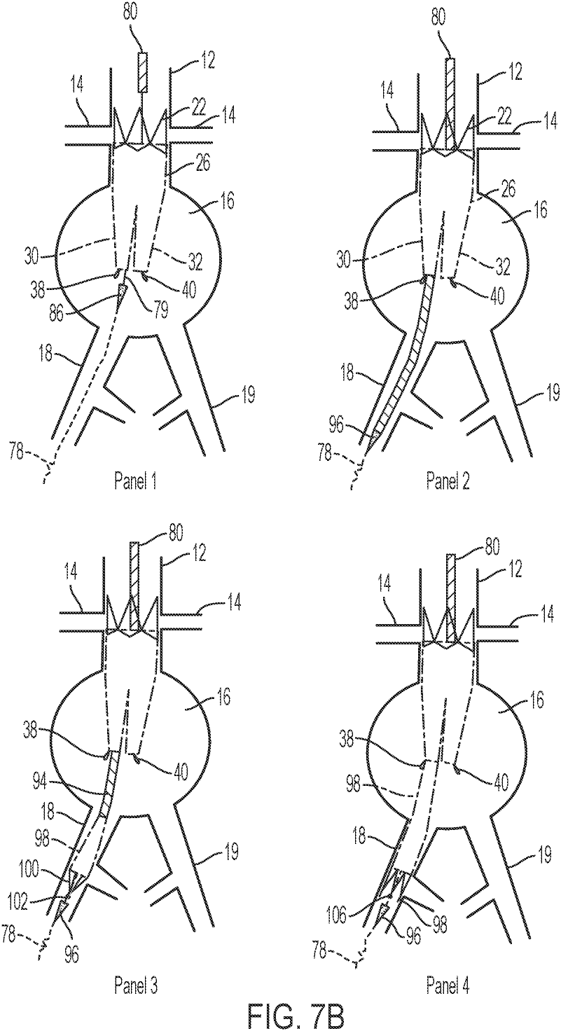

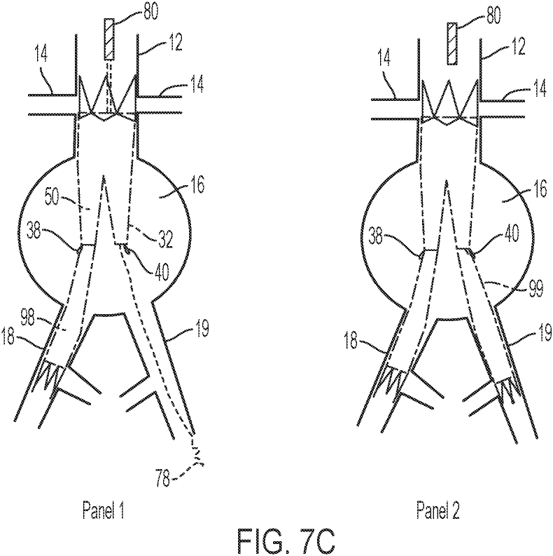

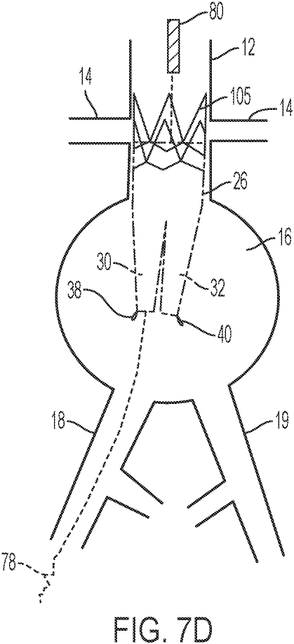

In contrast to the EVAR approach illustrated in FIGS. 6A and 6B, Panels 1 to 4 in FIG. 7A shows an exemplary insertion and deployment of the main body graft module from an insertion blood vessel located above the diaphragm of a patient.

In Panel 1, a puncture or incision is made in a blood vessel located above a patient’s diaphragm using a “top down” approach and delivery sheath 80 is inserted after removal of directional catheter (not shown). Guidewire 78 is inserted through inner member 79 and is shown advancing down suprarenal abdominal aorta 12 past renal arteries 14, across the aneurysmal infrarenal aorta 16 and into ipsilateral iliac artery 18 while contralateral iliac artery 19 remains uninstrumented.

In Panel 2, delivery sheath 80 containing/constraining main graft 26 (not shown) within its lumen is threaded over indwelling guidewire 78 via the lumen of the first nose cone 86 and subsequently introduced/inserted through said small artery puncture (vascular access point) and then advanced down the suprarenal abdominal aorta 12 past renal arteries 14, substantially across the aneurysmal infrarenal aorta 16 with first nose cone 86 positioned just above ipsilateral iliac artery 18 while contralateral iliac artery 19 continues to remain uninstrumented.

Panels 3 and 4 of FIG. 7A illustrate the deployment of delivery sheath 80 and initial delivery/release of main graft 26 in aneurysmal infrarenal aorta 16.

In Panel 3 of FIG. 7A, main graft body 26, first limb gate 30 and second limb gate 32 having radiopaque marker 88 are shown partially deployed and oriented both axially and rotationally as desired in the target anatomy. Radiopaque marker 88 acts as an aid in orienting the device under fluoroscopy. The partial deployment can be accomplished by partial retraction of delivery sheath 80 in the direction towards renal arteries 14 up suprarenal aorta 12 while generally maintaining a stationary position of main graft body 26 and inner member (not shown) having first nose cone 86 in aneurysmal infrarenal aorta 16. The position of guidewire 78 in ipsilateral iliac artery 18 is also maintained.

First tether 38 and second tether 40 are used to substantially fix the distal end of main graft body 26 to first nose cone 86 to permit traction and avoid crumpling or “riding up” of main graft body 26 during deployment. As previously noted, Panel 3 depicts partial deployment of the endograft by withdrawal (or proximal) retraction of delivery sheath 80.

As illustrated in Panel 4 of FIG. 7A, when delivery sheath 80 is fully retracted and main graft body 26 is released and self expands, with the exception that the proximal and distal ends of main graft body 26 remain constrained. This is achieved by fixedly constraining first tether 38 and second tether 40 on the distal end to first nose cone 86 using a tether wire (not shown) and using a similar system or arrangement (not shown) on first stent 22 on the proximal end. In this manner, crumpling of main graft body 26 is avoided during positioning and deployment..

FIG. 7B shows the deployment sequence for the right iliac limb component in a similar manner to the deployment of the main graft body. Panel 1 of FIG. 7B shows the main graft body fully released, including the top stent. Panel 2 of FIG. 7B shows the sheath has been advanced into the right external iliac artery. Panel 3 of FIG. 7C shows that the sheath has been partly withdrawn, partially releasing the right iliac limb gate. Panel 4 of FIG. 7B shows the right iliac limb gate is completely released.

In Panel 1 of FIG. 7B, main graft body 26, first limb gate 30, and second limb gate 32 are fully deployed by releasing the first tether 38 and second tether 40 from first nose cone 86 on the distal end and the fixation system on the proximal end on both the proximal and distal ends of main graft body 26 (i.e., first tether 38, second tether 40 and first stent 22 are no longer constrained). As shown in this panel, main graft body 26 is completely unconstrained and released from delivery sheath 80 with the stent graft now implanted in the vessel.

Panel 1 of FIG. 7B further depicts main graft body 26, in a state wherein it forms a substantially blood-tight seal (not shown) with aneurysmal infrarenal aorta 16. First stent 22 in Panel 1 of FIG. 7B is shown spanning renal arteries 14 without the covered portion of main graft body 26 blocking the perfusion or impeding the flow of blood into these important vessels. In addition, throughout the deployment maneuvers described in FIG. 7B Panels 1- 4, the position of guidewire 78 is generally maintained in ipsilateral iliac artery 18.

Upon completion of the full deployment of main graft body 26 in Panel 1 of FIG. 7B, the delivery system is carefully retracted and removed ensuring first nose cone 86 traverses the edge of ipsilateral leg component 98 during this maneuver. First nose cone 86 is attached to inner member 79. It is envisioned that first nose cone 86 may also be rendered radiopaque to ensure safe retraction of the delivery system from main graft body 26 via observation of the nose cone’s retraction with fluoroscopy during this procedural maneuver.

FIG. 7B (starting with Panel 2) and 7C illustrates exemplary positioning and deploying ipsilateral leg component 98 and contralateral leg component 106 (not shown) in an analogous manner to that described for the main graft body 26 deployment in FIG. 7A wherein delivery sheath 80 is withdrawn proximally while generally maintaining the relative stationary position of leg components during each of their respective deployment within main graft body 26.

In FIG. 7B, Panel 2, delivery sheath 80 is left in place and loaded with ipsilateral leg component 98 in constrained form through the puncture or incision in an insertion site blood vessel location above the diaphragm via delivery sheath 80, over guidewire 78, and through main graft body 26 and first limb gate 30 from a “top down” orientation, and positioned for unsheathing. Guidewire 78 is shown inserted through second nose cone 96.

In FIG. 7B, Panel 3, ipsilateral leg component 98 is partially unsheathed and finely positioned in ipsilateral iliac artery 18. The distal end of ipsilateral leg component 98 are constrained in a manner similar to that described in FIG. 7A, for example, by fixedly constraining tethers (not shown) on the distal end of ipsilateral leg component 98 to second nose cone 96 using a tether wire (not shown). At this stage, the proximal end of ipsilateral leg component 98 is still constrained in delivery sheath 80.

As shown in FIG. 7B, Panel 4, ipsilateral leg component 98 is fully unsheathed and deployed in ipsilateral iliac artery 18.

FIG. 7C, Panels 1 and 2 show the equivalent complete deployment (i.e., implantation) of contralateral leg component 99 in contralateral iliac artery 19 in the same manner as described in FIG. 7B. Panel 1 of FIG. 7C shows that guidewire 78 has been advanced into the left external iliac artery 19. Panel 2 of FIG. 7C shows a completely deployed contralateral leg component 99.

FIG. 7D shows an alternative where self-expanding additional stent 105 can be deployed in the sealing zone spanning the cranial graft edge to, for example, to further strengthen the substantially blood-tight seal.

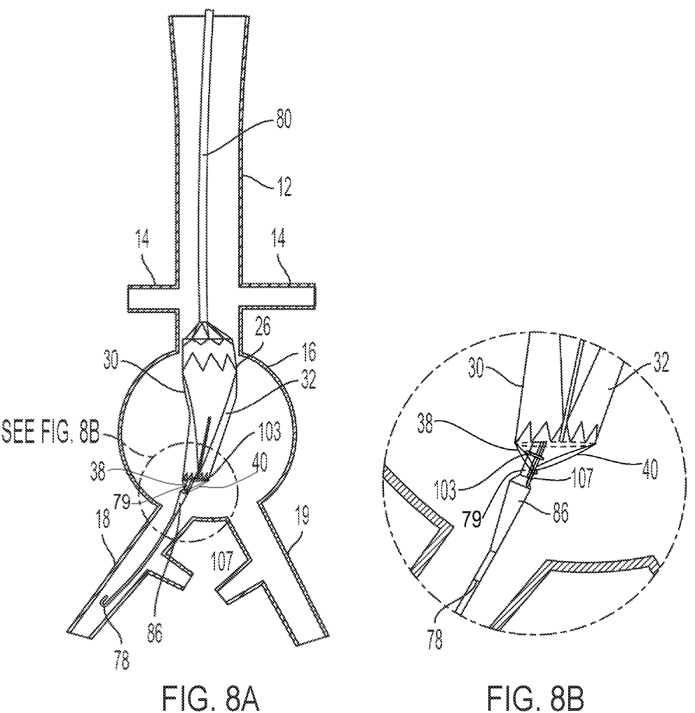

FIGS. 8A and 8B show an alternate deployment system in cross section (FIG. 8A) and close up (FIG. 8B). In this example, first tether 38 and second tether 40 are looped around and attached to tether wire 107 and around shelf 103 to control first limb gate 30, and second limb gate 32. First tether 38 and second tether 40 can be disposed anywhere around the circumference of first limb gate 30 and second limb gate 32, respectively. Shelf 103 is fixedly connected to the inner member 79. Retracting the tip of tether wire 107 from inside first nose cone 86 to a position up to or proximal to shelf 103 into the carrier catheter (not shown) releases first tether 38 and second tether 40 prior to deployment of main graft body 26.

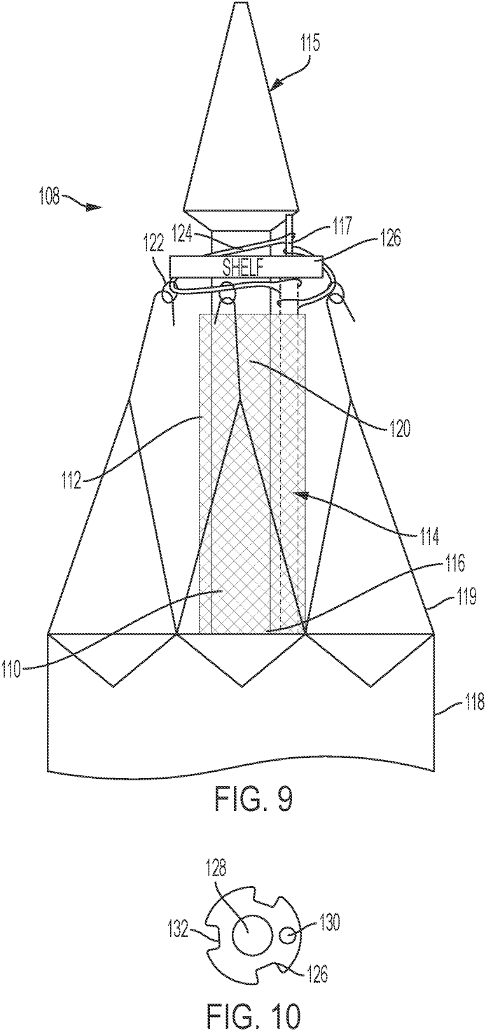

FIG. 9 shows an exemplary stent-graft deployment system 108 in accordance with the second stent-graft system for deploying an stent-graft 118 in the target blood vessel of a subject. The second stent-graft system described herein can be configured to be inserted in a femoral artery. The system includes outer tube 110 around central inner member 112 and carrier tube 114. Central inner member 112 has a caudad end with tip 115 and cephalad end 116 and is surrounded by stent-graft 118. Carrier tube 114 comprises tether wire 117. Tether wire 117 has a caudad end and a more cephalad portion.

Top stent 119 is shown surrounding the central inner member 112. Top stent 119 comprises a plurality of hooks 120 having a plurality of receptacles 122. In this example receptacles 122 are integral to hooks 120. Receptacles 122 can be, for example, spot welded to hooks 120. Hooks 120 can also be bent or arranged to function as receptacles. A plurality of sutures 124, wherein a first end of at least a first suture is disposed through one of the plurality of receptacles 122 for retaining the top stent 119 in a constrained configuration, and a second end of the first suture is affixed to the more cephalad portion of tether wire 117. An optional shelf 126 can be disposed around inner member 112 and carrier tube 114 for retaining carrier tube 114 and inner member 112 together.

As shown in FIG. 10, inner member 112 can be disposed through inner member hole 128 in shelf 126 and carrier tube 114 can be disposed through carrier tube hole 130 in shelf 126. Shelf 126 can function as a support for threading sutures 124 through receptacles 122 and for retaining inner member 112 and carrier tube 114 substantially together adding further radial stability to the system. Receptacle hole 132 can receive a portion of receptacles 122 with, for example, sutures 124 disposed through receptacles 122.

Movement of tether wire 117 can control removal of the plurality of sutures from the plurality of receptacles and release top stent 117 from a constrained to an unconstrained configuration. The plurality of sutures 124 can be removed from the blood vessel of the subject. In some instances, all of the sutures are removed from the blood vessel in order to, for example, protect the subject from negative side effects of sutures or portions of the sutures remaining in the subject for a period of time.

FIG. 11 illustrates an embodiment of the fourth stent-graft device including exemplary snare loop positioning features for adjusting the axial location of a main graft body in a target blood vessel. Outer sheath 134 encompasses an inner member 164 and snare tube 136. A centering device (not shown) is also encompassed by outer sheath 134. Snare loop 138 is disposed in and through snare tube 136 with an end that can be controlled by an operator. Snare loop 138 is shown threaded through a plurality of eyelets 140. Top stent 142 is shown with a plurality of hooks 144 disposed in eyelets 140. In the configuration shown, top stent 142 is in a constrained or closed configuration. Guidewire 160 is shown disposed through third nose cone 158.

Top stent 142 is shown in a substantially end-to-end configuration with main graft body 144. Main graft body 144 can be made of a densified material (e.g., densified ePTFE). Tether wire 146 is shown disposed through outer sheath 124 and main graft body 144 and ipsilateral limb gate 148 is shown as being connected to trigger wire holder 156. Contralateral tether 152 is shown disposed through contralateral limb gate 150 and attached to tether wire 146. Ipsilateral tether 154 is shown disposed through ipsilateral limb gate 148 and attached to tether wire 146. In this example, tether wire 146 can be used to adjust the position of contralateral limb gate 150 and ipsilateral limb gate 148 using contralateral tether 152 and ipsilateral tether 154.

Using the example of FIG. 11, an operator can insert the stent-graft device in, for example, a single puncture of a small artery located above the diaphragm and third nose cone 158 of the stent-graft device “top down” into the infrarenal aorta. In use, snare loop 138 can be retracted and pull top stent 142 into outer sheath 134 using hooks 144. The stent graft can optionally be repositioned axially in the blood vessel as desired by the operator. Releasing snare loop 138 can release top stent 142 in a deployed configuration once the stent-graft has been positioned in a desired location by the operator. In this exemplary manner, the position of the stent-graft can be adjusted and re-adjusted as needed. As shown in FIG. 11, snare loop 138 is retracted, top stent 142 is in a constrained configuration, and top stent 142 can be retracted into outer sheath 134. The position of the stent graft device can then be adjusted.

FIG. 12A illustrates an embodiment of the fourth stent-graft system. In this example, retraction of outer sheath 134 reveals centering basket 162. Snare loop 138 is not retracted and top stent 142 is deployed in an unconstrained configuration. Centering basket 162 can facilitate the centering and positioning of main graft body 144, for example, in the infrarenal aorta. The arms of centering basket 162 engage the walls of the suprarenal aorta when centering basket 162 is deployed by retracting outer sheath 134. In this manner, the location of the entire stent-graft apparatus can be adjusted with main graft body 144 centered, for example, in the infrarenal aorta. It is understood, as described herein, that centering basket 162 can be a centering device (e.g., basket, balloon or similar).

FIG. 12A depicts snare loop 138 in an unconstrained or released configuration which deploys top stent 142. If the operator wishes to adjust the position of main graft body 144, snare loop 138 can be tightened resulting in the re-constraining of top stent 142. Then, main graft body 144 can be re-positioned axially (in a cephalad or caudad direction) until a more desirable location in the infrarenal aorta is reached. Then, snare loop 138 can be loosened or released resulting in re-deployment of top stent 142 in a new location. The exemplary devices of FIGS. 11 and 12A show snare tube 136 in an asymmetrical configuration with respect to inner member 164.

In the examples of FIGS. 11 and 12A, contralateral tether 152 and ipsilateral tether 154 are shown as fixed to tether wire 146 such that tether 152 and tether 154 are not pushed upwards. Alternatively, contralateral tether 152 and ipsilateral tether 154 can be disposed in a catheter. The tether wire can exit the catheter just above the tether. The tether wire can be disposed through the center of the tether, back into the catheter through a hole in the catheter just below contralateral tether 152 and ipsilateral tether 154. In this manner, contralateral tether 152 and ipsilateral tether 154 can be pinned to the small length of wire outside its catheter. Contralateral tether 152 and ipsilateral tether 154 can be released when tether wire 146 is removed.

In a further alternate example, main graft body 144, top stent 142, and centering basket 162 are initially contained within outer sheath 134. In this aspect, when outer sheath 134 is removed from main graft body 144, top stent 142 can be deployed and centering basket 162 can be pushed axially in a caudad direction to the aortic neck and into main graft body 144. Centering basket 162 can be deployed inside main graft body 144 in the portion of main graft body 144 nearest to the heart. Centering basket 162 can then be retracted and snare loop 138 can be released to deploy top stent 142.

An alternate embodiment of the fourth stent-graft system is shown in FIG. 12B. FIG. 12B illustrates an exemplary alternative to the stent-graft device of FIG. 12A where snare loop 138 and snare tube 136 are disposed outside the perimeter of centering basket 162 but inside the perimeter of outer sheath 134. In this example, when centering basket 162 is pushed down, it will pass through the center of the snare loop 135, top stent 142, and into inner member 164.

Without being bound by theory, it is believed that sheaths used in blood vessels naturally tend to straighten along a straight line. When a sheath is inserted into a curved blood vessel, it can push against the wall of the vessel in an attempt to straighten. The force of the sheath against the vessel wall can depend on the material and its thickness. In one aspect, the wires or outer wall of an exemplary centering device (e.g., basket, balloon) is greater than the outward “straightening” force of a sheath such that the centering device does not collapse against the side of the blood vessel. In this manner, the stent-graft device can be centered in the blood vessel using the exemplary centering device.

In one aspect, the wires of a centering basket or the pressure exerted by the outer wall of a centering balloon exert enough force to hold the top stent in the center of the blood vessel against the force of the sheath. In another aspect, the force exerted longitudinally by the sheath is sufficient so that the sheath will not collapse on itself (e.g., this can be useful for advancing the sheath and for releasing the endograft). At the same time, the sheath can be flexible in terms of “side to side” movement but more rigid longitudinally to minimize the straightening force of the sheath. Additional flexibility for “side to side” movement can avoid a circumstance where the stent-graft device is pushed against the wall of the vessel and is not deployed symmetrically. In this aspect, endoleaks can be minimized.

FIG. 13 shows an instance of the fifth stent-graft system having a symmetrical configuration of snare loop 138 with respect to inner member 164. In this example, FIG. 13 illustrates a stent-graft device having top stent 142 with snare loop 138 threaded through eyelets 140 affixed above hooks 144 where snare loop 138 has a degree of rotation around top stent 142 of greater than 360 degrees. In one aspect, the degree of rotation can be from 360 to 800 degrees or a degree of rotation of about 540 degrees. In this manner, as shown in FIG. 13, snare loop 138 can disposed symmetrically with respect to inner member 164.

When snare loop 138 is used to retract top stent 142 into outer sheath 134 or deploy top stent 142 from outer sheath 134 in a desired location by an operator, top stent 142 is pulled inward symmetrically rather than being pulled to one side. In this manner, the stent-graft device can be maintained in a more centered configuration with respect to the target blood vessel.

In some instances, the bottom of the legs of the stent-graft device can be fixed to the introducer by trigger wires. The trigger wires can be used to move the entire endograft up and down in the aorta without crumpling the stent-graft device. The stent-graft device can be secured at both its top and its bottom between the lasso at the top and the trigger wires at the bottom (both can be released at final deployment).

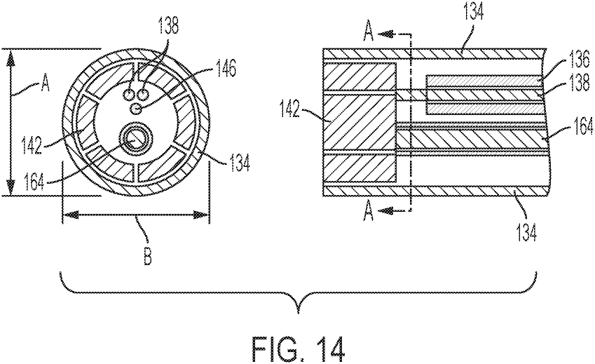

FIG. 14 illustrates cross sectional views of the aspect shown in FIGS. 11 and 12A showing snare loop 138 disposed in snare tube 136 asymmetrically with respect to inner member 164. As shown in the right panel of FIG. 14, snare tube 136 is shown to one side of inner member 164.

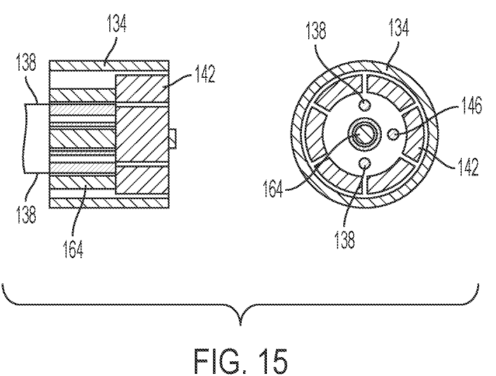

FIG. 15 provides exemplary cross sectional views of the aspect shown in FIG. 13 where the ends of snare loop 138 are disposed symmetrically with respect to inner member 164. As shown in FIG. 15, two strands of snare loop 138 are disposed on either side of concentric inner member 164 in a substantially symmetrical configuration.

In one aspect, the main graft body, first limb gate, and second branch graft comprise ePTFE (polytetrafluoroethylene). In another aspect, the ePTFE is an ultrathin composite made of, for example, up to 10 ply layers or more with layers as thin as about 0.00015ʺ. Sintering can be performed under high temperature with compression to adhere all ePTFE layers. In this aspect, the ePTFE has single direction strength with an orientation of layers that prevent creep. In this aspect, creep or movement of ePTFE should be avoided or minimized as it will allow for continuous expansion of the graft. In another aspect, the ePTFE can be configured to be impermeable to blood serum by the addition of, for example, an FEP (fluorinated ethylene propylene) layer(s), in order to provide a substantially blood tight seal. In a further aspect, the total thickness of the ePTFE as sintered is about 0.0015ʺ.

In some instances, the main graft body, first limb gate, and second limb gate can be made of human or animal tissue or artificial tissue. See, e.g., Deeken et. al., Differentiation of Biologic Scaffold Materials Through Physicomechanical, Thermal, and Enzymatic Degradation Techniques. Annals of Surgery, March 2012; U.S. Pat. Application Pub. No. US20180326120.

While the aspects described herein have been disclosed with reference to certain embodiments, numerous modifications, alterations, and changes to the described aspects are possible without departing from the sphere and scope of the present disclosure, as defined in the appended claims. Accordingly, it is intended that the present disclosure not be limited to the described aspects, but that it has the full scope defined by the language of the following claims, and equivalents thereof.

1-139. (canceled)

140. A stent-graft system for repair of an aneurysm in a target blood vessel of a patient comprising: a main graft body having a first end and a second end; at least one stent connected to and/or disposed at least partly with the main graft body; and a delivery sheath to position the main graft body and the at least one stent to a location in the target blood vessel in proximity to the aneurysm, the delivery sheath being oriented relative to the main graft body and the at least one stent so that removal of the delivery sheath from the target blood vessel initially exposes the second end of the main graft body and subsequently exposes the first end of the main graft body, wherein the main graft body is configured to be inserted through a single arterial puncture or incision in an insertion site blood vessel located above a diaphragm of the patient.

141. The stent-graft system of claim 140, further comprising a centering device in proximity to the first end of the main graft body and configured to be removably or temporarily deployed and expanded in the target blood vessel for centering the main graft body and/or the at least one stent in the target blood vessel.

142. The stent-graft system of claim 140, wherein the at least one stent comprises a first stent adjacent the first end of the main graft body and a first tether releasably connected to the first stent for positioning the first stent and the first end of the main graft body in the target blood vessel.

143. The stent-graft system of claim 140, wherein the at least one stent comprises a first stent adjacent the first end of the main graft body, the stent-graft system further comprising: a connecting ring in the main graft body at a position substantially adjacent the first end of the main graft body, and connecting members connecting the connecting ring to the first stent.

144. The stent-graft system of claim 140, wherein the at least one stent comprises a first stent adjacent the first end of the main graft body, and the stent-graft system further comprising a snare tube passing through the delivery sheath and a snare loop passing through the snare tube, the snare loop being releasably engaged with a plurality of circumferentially spaced positions on the first stent and being operative for selectively adjusting the first stent from an unconstrained circumferentially expanded condition to a constrained circumferentially collapsed condition so that the first stent and at least parts of the main graft body adjacent to the first stent can be positioned or repositioned in the target blood vessel.

145. The stent-graft system of claim 140, wherein the second end of the main graft body is bifurcated.

146. The stent-graft system of claim 145, wherein the bifurcated second end of the main graft body comprises a first limb and a second limb, a first limb stent and a second limb stent being disposed respectively in the first limb and the second limb of the main graft body.

147. The stent-graft system of claim 146, further comprising an inner member passing from the delivery sheath, through the main graft body and to a position beyond the first and second limbs, a wire holder connected to the inner member at a position beyond the first and second limbs and first and second limb tethers connected respectively to the first and second limb stents.

148. The stent-graft system of claim 140, wherein the main graft body comprises a densified material.

149. The stent-graft system of claim 148, wherein the densified material is ePTFE.

150. The stent-graft system of claim 140, wherein the at least one stent is encapsulated in a densified material.

151. The stent-graft system of claim 150, wherein the densified material is ePTFE.

152. An endograft deployment system for deploying an endograft in a target blood vessel of a subject, comprising:
an outer tube comprising a central inner member comprising an endograft and a carrier tube comprising a tether wire, the tether wire having a caudad end and a more cephalad portion;
a top stent surrounding the central inner member, wherein the top stent comprises a plurality of hooks having a plurality of receptacles; and
a plurality of sutures, wherein a first end of at least a first suture is disposed through one of the plurality of receptacles for retaining the top stent in a constrained configuration, and a second end of the first suture is affixed to the more cephalad portion of the tether wire; and
wherein movement of the tether wire can control removal of the plurality of sutures from the plurality of receptacles, release the top stent from a constrained to an unconstrained configuration, and remove the plurality of sutures from the blood vessel of the subject.

153. The endograft deployment system of claim 152, wherein the first end of the at least a first suture is formed into a loop, and the loop is disposed around a caudad end of the tether wire.

154. The endograft deployment system of claim 152, further comprising a shelf disposed below the first end of the at least a first suture for initially retaining the first end of the at least a first suture above the shelf and retaining a second end of the at least a first suture below the shelf.

155. The endograft deployment system of claim 154, wherein the shelf further comprises a first opening for receiving the central inner member.

156. The endograft deployment system of claim 155, further comprising a second opening for receiving the carrier tube.

157. The endograft deployment system of claim 156, further comprising a third opening for receiving at least one of the plurality of sutures.

158. A method of repairing an abdominal aortic aneurysm in a patient, comprising:
puncturing an insertion site blood vessel located above a diaphragm of the patient and
creating a passage in the insertion site blood vessel;
inserting a stent-graft system in a passage of the insertion site blood vessel, the stent-graft system comprising a first stent, a main graft body, a first limb gate and a second limb gate;
positioning the main graft body of the stent-graft system in a target blood vessel of the patient; and
deploying the main graft body of the stent-graft system in target blood vessel of the patient.

159. The method of claim 158, wherein the stent-graft system further comprises a first iliac leg component and a second iliac leg component, and wherein the method further comprises:
positioning and deploying the first iliac leg component in a first branch of an iliac artery; and
positioning and deploying the second iliac leg component in a second branch of an iliac artery.

160. The method of claim 159, further comprising using a guide wire to position the main graft body in the target blood vessel in a constrained state.

161. The method of claim 160, further comprising partially unsheathing the main graft body for further positioning the main graft body in the target blood vessel.

162. The method of claim 161, further comprising unconstraining the main graft body after the main graft body is deployed in the target blood vessel.

163. The method of claim 162, further comprising deploying a sealing stent in the main graft body.

164. The method of claim 159, using a guidewire for positioning the first iliac leg component in the first branch of the iliac artery in a constrained state through the main graft body.

165. The method of claim 164, further comprising partially unsheathing the first iliac leg component for further positioning the first iliac leg component in the first branch of the iliac artery.

166. The method of claim 165, further comprising unconstraining the first iliac leg component after the first iliac leg component is deployed in the first branch of the iliac artery.

167. The method of claim 166, using a guidewire for positioning the second iliac leg component in the second branch of the iliac artery in a constrained state through the main graft body.

168. The method of claim 167, further comprising partially unsheathing the second iliac leg component for further positioning the second iliac leg component in the second branch of the iliac artery.

169. The method of claim 168, further comprising partially unsheathing the second iliac leg component for further positioning the second iliac leg component in the second branch of the iliac artery.

170. The method of claim 158, wherein the stent-graft system further comprises a connecting ring, wherein the first stent comprises a plurality of receptacles and the connecting ring comprising a plurality of connectors adapted to receive the plurality of receptacles, wherein the connecting ring is disposed in the main graft body, and the first stent and the main graft body are in a substantially end-to-end configuration.
